(19) United States
(12) Patent Application Publication
(10) Pub. No.: US 2026/0120811 A1
(43) Pub. Date: Apr. 30, 2026

(54) Process for Practicing Fractal Science using KFE

(71) Applicant: Gregory M. Friedlander, Mobile, AL (US)

(72) Inventor: Gregory M. Friedlander, Mobile, AL (US)

(21) Appl. No.: 18/921,220

(22) Filed: Oct. 21, 2024

Related U.S. Application Data
(63) Continuation-in-part of application No. 18/425,251, filed on Jan. 29, 2024.
(63) Continuation-in-part of application No. 18/196,930, filed on May 12, 2023.
(63) Continuation-in-part of application No. 16/880,255, filed on May 21, 2020, now abandoned.
(63) Continuation-in-part of application No. 16/670,029, filed on Oct. 31, 2019, now abandoned.
(63) Continuation-in-part of application No. 16/552,481, filed on Aug. 27, 2019, now abandoned.
(63) Continuation-in-part of application No. 16/530,339, filed on Aug. 2, 2019, now abandoned.
(60) Provisional application No. 62/723,765, filed on Aug. 28, 2018.
(60) Provisional application No. 62/741,025, filed on Oct. 4, 2018.
(60) Provisional application No. 62/750,927, filed on Oct. 26, 2018.
(60) Provisional application No. 62/771,044, filed on Nov. 24, 2018.
(60) Provisional application No. 62/787,982, filed on Jan. 3, 2019.
(60) Provisional application No. 62/798,573, filed on Jan. 30, 2019.
(60) Provisional application No. 62/851,832, filed on May 23, 2019.
(60) Provisional application No. 62/870,352, filed on Jul. 3, 2019.
(60) Provisional application No. 62/910,889, filed on Oct. 4, 2019.
(60) Provisional application No. 62/956,989, filed on Jan. 3, 2020.
(60) Provisional application No. 62/967,891, filed on Jan. 30, 2020.
(60) Provisional application No. 63/018,056, filed on Apr. 30, 2020.
(60) Provisional application No. 63/071,190, filed on Aug. 27, 2020.
(60) Provisional application No. 63/106,064, filed on Oct. 27, 2020.
(60) Provisional application No. 63/145,752, filed on Feb. 4, 2021.
(60) Provisional application No. 63/162,147, filed on Mar. 17, 2021.
(60) Provisional application No. 63/191,149, filed on May 20, 2021.
(60) Provisional application No. 63/218,226, filed on Jul. 2, 2021.
(60) Provisional application No. 63/240,646, filed on Sep. 3, 2021.
(60) Provisional application No. 63/305,415, filed on Feb. 1, 2022.
(60) Provisional application No. 63/313,815, filed on Feb. 25, 2022.
(60) Provisional application No. 63/343,161, filed on May 18, 2022.
(60) Provisional application No. 63/399,286, filed on Aug. 19, 2022.
(60) Provisional application No. 63/402,977, filed on Sep. 1, 2022.
(60) Provisional application No. 63/422,041, filed on Nov. 3, 2022.
(60) Provisional application No. 63/436,647, filed on Jan. 2, 2023.
(60) Provisional application No. 63/441,856, filed on Jan. 30, 2023.
(60) Provisional application No. 63/451,300, filed on Mar. 10, 2023.
(60) Provisional application No. 63/545,185, filed on Oct. 22, 2023.
(60) Provisional application No. 63/635,110, filed on Apr. 17, 2024.

(51) Int. Cl. G16C 10/00; G16C 60/00

(52) U.S. Cl. CPC G16C 10/00; G16C 60/00

(57) ABSTRACT

The present invention relates to a method for improving processes in various undertakings by employing Key Fractal Elements (KFE). The method involves utilizing KFE to affect change in CT states and interpret matrices of CT states. By applying KFE elements, categorization, prediction, manipulation, and the design of radiation matrices for electronics, solar, thermal, fusion, and radioactive energy applications are achieved. By incorporating KFE, improved balance, plasma fulcrums, and efficient CT state interactions are achieved, leading to advancements in various fields. The application of KFE in reaction processes, fuel utilization, electronics, and energy management contributes to superior matrix changes and efficient pretime informational exchange.

### PRIOR ART

The prior art is forced to create intuitive models instead of math-based models.

### THE INVENTION

### Field of the Invention

The present invention generally relates to the field of artificial intelligence and data processing, and more particularly to a process for practicing fractal science using a knowledge fusion engine.

### Background of the Invention

The field of the invention pertains to the integration of artificial intelligence with scientific data analysis, specifically focusing on the application of fractal science. The current scientific landscape is characterized by inconsistent data terminology, varied languages, and fragmented data structures across multiple disciplines such as physics, chemistry, biology, and engineering. These inconsistencies hinder the efficient extraction and analysis of knowledge, posing significant challenges for AI systems to work effectively with scientific data.

Existing solutions in the prior art have attempted to address these issues by creating intuitive models for data analysis. These models often rely on heuristic or empirical approaches rather than rigorous mathematical frameworks. While some progress has been made, these solutions fall short in providing a comprehensive and unified method for handling the diverse and complex nature of scientific data across different fields.

The limitations of the current approaches are manifold. Intuitive models lack the precision and reliability of math-based models, leading to potential inaccuracies in data interpretation and analysis. Additionally, the fragmented nature of data structures and terminologies across disciplines further complicates the integration and synthesis of scientific knowledge. These drawbacks significantly impede the advancement of scientific research and the development of innovative solutions.

In light of these deficiencies, there is a pressing need for a more effective method to practice fractal science using a Knowledge Fusion Engine (KFE). The proposed invention, titled “Process for Practicing Fractal Science Using Knowledge Fusion Engine,” aims to address these challenges by providing a robust and unified approach to scientific data analysis, thereby enhancing the efficiency and accuracy of AI systems in this domain.

In the context of the present disclosure, it should be understood that the described embodiments in this section are put forth for illustrative purposes only. Those skilled in the art will appreciate that various modifications, adaptations, and alternative designs may be employed without departing from the scope and spirit of the invention.

Accordingly, the present invention should not be limited to the specific embodiments illustrated herein, but rather should be construed according to the claims and description that follow.

### OBJECTS OF THE INVENTION

An object of the invention is to provide a method for substituting different languages with a single underlying mathematics, thereby enabling a more efficient and unified approach to modeling and understanding various systems and processes.

Another object of the invention is to provide a method for translating any desired outcome into the FIP model, allowing for the most efficient execution of processes by leveraging the new model's capabilities.

Yet another object of the invention is to provide a method for converting prior art modeling into FIP modeling, thereby enabling a more efficient understanding and modeling of features through the new perspective offered by the FIP model.

An additional object of the invention is to provide a method for using fractals with data science to modify AI software, thereby maximizing the accessibility, organization, and value of empirical data, and enabling the use of this data along with fractal modeling to predict chemical and biological functions.

A further object of the invention is to provide a fractal model applicable across energy, atomic, chemical, and biological systems, allowing for the categorization of data from sub-atomic through molecular systems up to and including complex biological systems and astronomical features using the same modeling framework.

Another object of the invention is to develop a classification system that cross-characterizes existing empirical non-fractal data within sub-atomic, atomic, chemical, and biological domains with the fractal modeling required by the “FIP” (formally AuT) Fractal modeling developed by the inventor.

Yet another object of the invention is to integrate the fractal model into AI software, enabling the AI software to access, organize, and analyze empirical data more effectively.

An additional object of the invention is to use AI software integrated with the fractal model to predict chemical and biological functions, thereby enhancing the accuracy and efficiency of such predictions.

A further object of the invention is to provide a method for modeling atomic interactions for energy transfer, utilizing fractal analysis techniques to identify patterns in empirical data and develop fractal atomic structure definitions and mathematical models that describe transitions between energy and matter.

Another object of the invention is to provide a system for automated fractal analysis of empirical chemistry data, comprising a database, a software module, and a processor configured to execute the software module, thereby enabling the extraction, categorization, and simulation of data based on fractal constraints.

Yet another object of the invention is to improve processes in various undertakings by utilizing Key Fractal Elements (KFE) to affect change in CT states, interpret or categorize matrices of CT states, and optimize reactions and energy processes across multiple applications, including electronics, solar, thermal, fusion, and radioactive energy use.

An additional object of the invention is to provide methods for improving fusion and fission processes, chemistry processes, polymerization reactions, hydrogen extraction reactions, and the design of electronic devices and circuits, quantum computing systems, and AI algorithms and models, all by leveraging Key Fractal Elements (KFE) to enhance efficiency, control, and performance.

A further object of the invention is to provide a system for controlling compression and decompression of CT states within at least one AuT matrix or between multiple AuT matrices, utilizing key fractal elements to govern CT state changes, absorption, and spew mechanisms, and achieve desired dimensional variations.

Another object of the invention is to utilize key fractal elements in the creation of new materials and substances by controlling the interaction and fusion of CT states within at least one AuT matrix, thereby enabling the design and production of materials with unique properties and characteristics.

Yet another object of the invention is to model and simulate complex systems and phenomena by controlling the interaction and fusion of CT states within at least one AuT matrix, providing a new method for simulating and predicting the behavior of complex systems.

### APPLICATIONS OF FIP MODELING

The process is taking any desired outcome and translating the elements into the FIP model and then carrying out the process most efficiently by virtue of the new model;

taking prior art modeling, translating it into FIP modeling and then taking advantage of the new view to understand and model features efficiently. Non fractal terms like fission, fusion and even computing can all be replaced with the fundamental process at the core of these terms.

This maximizes the efficiency of any reactions and the absorption or release (referred to as spew in earlier patents), and control of information redefined along the specifics of Fractal Information Physics (FIP, previously AuT).

The iterated equations: 1) Fpix, ubiquitously observed in curvature, 2) MI, observed above the level of the neutron and compression of fpix and MI based on 2f(n)\hat{ }(2\hat{ }n) where f(n) for electrons and protons is a function of fpix and a function of MI for neutron counts. For f(x)=fpix, where n=4, f(n)=7 and 2\hat{ }n=16. This allows the equation of 2f(n)\hat{ }16 to be viewed as 2 sets of 7 units doubling 16 times.

The Rule of 4's is seen with Resonance defined by fpix modeling to the inverse of fpix modeling based on how it gives rise to curvature in FIG. 2 which shows the intersection of fpix to x/fpix as x changes as shown.

### KEY FRACTAL ELEMENTS

This application covers the use of KFE in fusion, fission, chemistry, polymer chemistry, hydrogen extraction, electronics, batteries, quantum computing, and AI. Application of KFE involves interpreting and categorizing matrices of CT states and utilizing FIP to bring about changes in CT states.

Base FIP transitions governing matrix composition and interaction, including spatial, energy, atomic, chemical, electrical, biological, and large structures, are KFE. KFE incorporates concepts such as CT states, fpix equations, fuse length, MI, defined spirals, overlapping and exponential compression features, pretime change characteristics, spiral alignment, CT state sharing, collisions as information exchange, net compression/decompression, balance and imbalance, changing base numbering, and net pretime change characteristics. Reacting chemicals using KFE enhances matrix changes, maximizes the release of pretime informational change, and optimizes the use of energy states.

Balance about fulcrums is reflected in charting of resonance discussed below. This is a transitional step in compression and creating fulcrums of information, a KFE, observed in atomic and molecular structure about the lower ct state or plasma fulcrum, folding patterns, magnetic repulsion effects, and high- and low-pressure system interactions.

Dimension building can be exemplified with pi(4/sqr(6)=r\hat{ }2 reflecting the location where dimension shifts. A similar shift should be capable of calculation using the equation for the volume of a circle is 4/3pir\hat{ }3. Calculate V with r*4/3pi (4/sqr(6))*sum(fpix(4)) is complex because of shifts in underlying curvature, from pi(4/sqr(6)) to pi(4). KFE provides targets where the process are applied. Application of KFE is using KFE to affect change in CT states, interpret or categorize matrices of CT states and to design and then manipulate matrices.

KFE includes CT states, fpix as an equation, fuse length for individual solutions to fpix as well as for matrices of solutions and compression states, MI, MI defined spirals, overlapping and exponential compression features, particularly as functions of fpix and MI, changing vs stable pretime features, the nature of pretime change vs time-based change, the relative amount of pretime change vs non change in a matrix, changes in the quantum count, KFE spiral alignment, pretime change CT state changes, CT state sharing and the related concept of collisions as information exchange, net compression/decompression of CT states and matrices and the related force or energy features, balance and imbalance, changing base numbering reflected in CT states and dimensions, and net pretime change characteristics or a combination of those features. KFE can be used in field modeling and mechanical interactions.

KFE includes changes in KFE and KFE energy converters can be used for staged fuels with fpix, MI and exponential changes such as 1:1; 1:2: 1:3:1:5 for burn times, heat, pressures, mixtures, steps and reactant matrices beginning and ending for any reaction to target specific fractal results.

KFE includes and enables dimensional variations in a matrix and the benefits that come with work with dimensional variation. KFE includes control of where absorption and spew are generated within matrices of CT states and where it is discharged into matrices which includes design of machines, reactions using KFE.

FIP modeling allows for time to be taken out of equations, replaced with pretime change to reduce variables. KFE includes targeting AuT plasma acting as fulcrums and associated stepped transitions. The base structure of fulcrum likely first appears as a result of Resonance. Balance is shown where a plasma fulcrum is the central most neutron-to-neutron plasma fulcrum of shared information between those central most neutrons, everything else balanced and folding around the fulcrum; balanced by absorption and spew between the elements. A fulcrum of lower states about which higher states balance, fractal alignment on either side of the fulcrum occurs because higher CT states form within shells of folding lower states. Balance and folding around the fulcrum are by absorption and spew between the neutrons and then between the protons and neutrons and then between the electrons and protons. This fulcrum of lower CT states continues, including the effects of magnetic repulsion and their corresponding effects of high- and low-pressure system interactions.

### GROUPING OF KFE

### Key Fractal Elements Observed:

CT states and resonance.

Base transitions governing matrix composition and interaction in various domains.

Compression and decompression of CT states and formation of matrices.

Fractal features such as spirals, overlapping, and exponential compression.

Pretime change and its relationship to dimensional variation.

Spiral alignment and its impact on CT state changes.

CT state sharing and collisions as information exchange mechanisms.

Net compression and decompression of CT states and matrices.

Balance and imbalance within fractal structures.

Changing base numbering and resulting dimensions.

Pretime change characteristics and their effects on matrices.

KFE's influence on quantum computing and AI algorithms.

These elements represent key observations and principles related to fractal phenomena and their application in various domains, as described in the disclosed information. It is helpful to think of KFE groups.

### Group 1: Fractal Elements of CT State Transitions

Stepped AuT fractal transitions are defined as fractal transitions governing CT state changes. MI overlapping spirals of CT states in and out of alignment and related absorption and spew between matrices. Pairing of higher compression CT states along f-series linear spirals, curved spirals and shared lower compression CT states.

Folding to get compression and unfolding to get decompression along fractal linear spirals of CT states about at least one AuT fulcrum of “shared” lower CT states, shared meaning that over time relevant quantum changes CT states from the fulcrum are exchanged with the higher CT state matrices on either side.

Compression as lowering lower compression CT states between higher compression CT states and decompression as numerically increasing lower compression CT states between higher compression CT states.

Fulcrums as AuT plasma centers between CT states defined as the areas between CT states where lower CT states are shared by higher CT states along KFE rules of compression. Net compression or decompression as force when observed from the standpoint of time. CT states defined as stepped fractal dimensional states from iterated equations defined by fractal compression or decompression due to compression of lower CT states along KFE rules of compression.

Force defined as the result of net winding or unwinding of CT states as viewed from post-time CT state perspectives including ct4t11 pretime changes viewed as energy.

Time is defined as stop-frame animation resulting from changes in pretime CT states defined as CT states at and below the level generating electromagnetic effects.

Relativistic effects as the difference between pretime and time-based change.

Curvature defined by a solution to fpix for pi with definitive limitations generating the sequential amounts of dimension and curvature in response to net CT state compression.

Group 2: Elements related to AuT matrices.

Base state changes are inherent in CT state folding.

Fractal balance, defined as alignment of at least two higher compression CT states about at least one AuT fulcrum comprised of lower compression CT states by way of sharing the fulcrum.

AuT fulcrums are defined as lower compression CT states at the overlap of compression of at least two higher CT states; where a ct4 state neutron backbone is part of the higher CT states to form atoms.

Absorption can increase or decrease compression of a matrix to higher CT states. It depends on the net compression of the matrix at the two points of observation. The same is true about spew of lower states from within a higher CT state compression matrix.

Using KFE to define “base logic,” mathematical “base” term use, to categorize at least one AuT Matrix. Key fractal elements (KFE) define the formation and manipulation of pretime. KFE encompasses a range of mathematical principles, structural patterns, and dimensional features that govern the behavior of particles, systems, and phenomena.

These elements include concepts such as fractal spirals, balance and imbalance, dimensional variation, energy exchange, and the intricate interplay between different CT states, net compression as the net compression or decompression within an AuT matrix; shifting between higher and lower compression of CT states within the AuT fulcrum; Absorption and spew between at least the first AuT matrix and at least one second AuT matrix which is a little different where the second matrix includes the first within it; CT state exchange between at least two AuT Matrices in place of collision or field modeling.

AuT matrix categorization using 1) CT state content, 2) amount of CT states within the content; 3) relative dimensional size to at least one second matrix, 3) locational area from the perspective of time of generated by pretime change of the at least one matrix;

4) AuT plasmas; 5) fulcrum locations; and the amount of pretime change of the CT states, compression and decompression tendency within a matrix.

Basing thermodynamic effects of at least one AuT matrix based on categorized CT states within the at least one AuT matrix.
Group 3: Absorption and spew of CT states
Treating exchange of lower CT states between at least two higher CT state AuT matrices as the source of interaction

Collisions as the exchange of information between at least two AuT matrices and post-collision effects reflecting the net change of CT state and pretime change in each matrix of the at least two matrices.
F-series spirals of CT states in and out of alignment for absorption and spew
Group 4: CT states and fractal elements

CT states are defined as stepped fractal dimensional states from common iterated equations defined by fractal compression or decompression due to compression of lower CT states along f-series fractal lines.

Force defined as the result of net winding or unwinding of CT states as viewed from post time CT state perspectives including ct4t11 changes viewed as energy.

Time defined as stop frame animation resulting from changes in pretime CT states defined as CT states below the level generating electromagnetic effects; relativistic effects as the difference between pretime and time-based change.

Fuse length as a fractal element of CT state transition from compression to decompression.

Curvature defined by a solution to fpix for pi with definitive limitations generating the sequential amounts of dimension and curvature in response to net CT state compression.

Fulcrums as shared CT states between higher CT states.

Quantum fractal dimensional change in place of divisions of time which is more fluid since it is an Effect of quantum changes at a relatively high level of compression.

Group 5: AuT matrices and their categorization

Using AuT as “base logic” of at least one AuT Matrix.

Net compression as the net compression or decompression along a fractal pathway mathematically derived from fpix, quantum changes in solutions to fpix, and ultimately the components of fpix within an AuT matrix.

Shifting between higher and lower compression of CT states within the AuT fulcrum

Absorption and spew between at least the first AuT matrix and at least one second AuT matrix

AuT matrix categorization using 1) CT state content, 2) organization and type of CT states leading to compressive features within the matrix; 3) locational area from the perspective of time; 4) energy level generated by pretime change of the at least one matrix; and 5) fulcrum locations and categorization. This includes basing related thermodynamic properties of a first AuT matrix with at least one second AuT matrix based on the categorized matrices and intervening CT state matrices.

CT state exchange between at least two AuT Matrices in place of collision or field modeling

Visible at the Proton-Neutron interface is balance of f-series linear spirals, net absorption leading to the folding or closing in of the spirals around the areas of overlap represented by fulcrums which incrementally increases the compression.

Imbalance using lower information states. The direction and form of winding and to maximize CT state exchanges to transfer information and maximize work.

Key Fractal Elements (KFE) can be used to improve the performance of a variety of technologies.

KFE can be used to improve the efficiency of energy conversion, the accuracy of signal processing, and the stability of quantum systems. KFE enables increased energy efficiency, improved energy capture and dispersal, and enhanced energy management techniques, enhances reaction kinetics, allows for a way for CT states to be interpreted in a manner that increases reaction selectivity; optimize fission in a manner that improves product yields; allows for the design of molecules and polymer structure formation and improves polymer properties. KFE can be used to interpret or categorize matrices of CT states; for categorization, prediction, manipulation, and designing radiation matrices for various applications including electronics, solar, thermal, fusion, and radioactive energy use, capture, and dispersal; to modify frequency-based systems, thereby increasing the efficiency of energy generation, transmission, utilization, and storage, while enhancing overall accuracy of results; to leverage base transitions as KFE elements to govern matrix composition and interaction, encompassing spatial, energy, atomic, chemical, electrical, biological, and large structures, applicable across a range of CT states; to efficiently compress or decompress CT states and form matrices of CT states, facilitating desired dimensional variations; to design matrices based on KFE concepts, considering time as CT state dimensional change, energy as CT state change, and the structural aspects of neutron backbones, proton cores, and electron clouds, enabling control over energy release, redirection, and absorption; and design includes the design and manipulation of molecules, including the expansive or contractive features and orientation of atomic or molecular matrices throughout reactions as CT state matrix change occurs; to control the absorption and spew of CT state exchanges within matrices by strategically shaping reaction chamber parts, adjusting injector jets, and exhaust systems; to react chemicals, including fuels, for enhanced matrix changes, maximizing the release of pretime informational change, and optimizing energy utilization and to target AuT plasma, stepped transitions, categorization, chemistry, biology, and other relevant features, thereby improving processes in any undertaking.

The remarkable potential of key fractal elements (KFE) to revolutionize various industries cannot be overlooked. Through the integration of KFE in fusion, fission, chemistry, polymer chemistry, hydrogen extraction, electronics, batteries, quantum computing, and AI, we can achieve unprecedented advancements and reshape the landscape of these fields. The presented argument for the issuance of a patent for the application of KFE highlights the transformative impact it can have on energy generation, materials science, computational systems, and more. Embracing KFE unlocks a new era of innovation, sustainability, and efficiency, shaping a future where the boundaries of what is possible are pushed ever further.

### IN THE DRAWINGS

FIG. 1 shows representative features for Fission Modeling

FIG. 2 shows a representative view of ct3 to ct4 compression.

FIG. 2a shows how energy is represented by pretime change of particles.

FIG. 2b shows the relative compression sizes in base 14 (2*7).

FIG. 2c shows electron structure in base 14.

FIG. 2d shows a view of plasma modeling.

FIG. 3 shows a representative view of magnetism from electricity based on fractal modeling and associated magnetic attraction.

FIG. 4 shows a representative view of magnetic repulsion based on fractal modeling.

FIG. 5 shows a representative view of the growing proton count for protons based on fractal modeling.

FIG. 6 shows a representative view of carbon based on fractal modeling.

FIG. 7 shows a representative view of a larger atom neutron count based on fractal modeling.

FIG. 8 shows a representative view of neutron bonding as sets of carbon like base structures.

FIG. 9 shows the carbon and argon neutron features giving rise to the structures shown in FIG. 8.

FIG. 10 shows a view of argon from FIG. 9 including protons and electrons, at least in terms of equivalence.

FIG. 11 Shows bonding represented from fractal modeling of atoms.

FIG. 12 shows a representative view of hydrogen separation using fractal modeling.

FIG. 13 shows a representative view of water using fractal modeling.

FIG. 14 shows fusion modeling using fractal modeling.

FIG. 15 shows a side view of the model shown in FIG. 15.

FIG. 16 shows a representative view of a virtual chamber used in FIG. 15.

FIG. 17 shows a representative view of one form of ct1 solution folding.

FIG. 18 shows process steps to carry out fractal modeling of existing science and to use that in further design.

FIG. 19 shows a view of different fractal transitions within resonance as shown in FIG. 1.

FIG. 20 shows where the lengths for neutron count can be found in fractal modeling of neon.

FIG. 21 shows modeling of resonance in a chart.

### SPECIFICATION

A fractal algorithm-based universe must have base equations which are ubiquitous. For clarity, we begin with fpix, the equation giving rise to the denominator of pi, the ubiquitous phenomenon incorporating the equation is curvature. The details are important, but the nature of Key Fractal Elements (KFE) which give us control over features of the universe using the new modeling, including the precise transitions will be developed over time. The process of using KFE to interpret and control interactions with a universe driven by pretime change can be identified without all of the refinements which will come about. Pretime refers to the dimensional changes which occur which 9 give rise to time as stop frame animation.

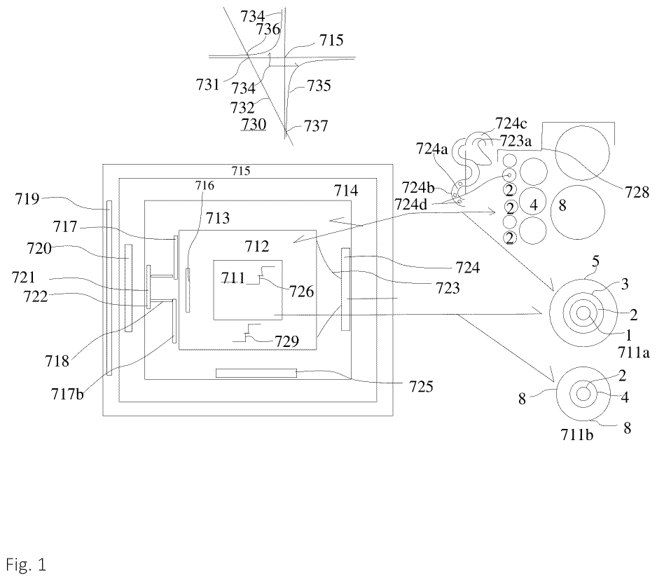

At the levels where we live, there are at least three choices, Fibonacci (1,2,3,5,8,13), fpix(1,3,5,7,9) and the building sum(1,2,5,8,3,2,7,12) which results from the absolute value of “fused” values of fpix over time as shown in FIG. 1. An analysis of observations shows that all of these solutions to f(x) in the iterated equation CTx=f(x)\hat{ }2\hat{ }x evolve and devolve as is the case for this unique fractal, fpix which must itself evolve from and according to the more base fractal leading to the quantum count.

$${f}_{p i} ( x ) = - {1}^{x} + 2 x * - {1}^{x - 1}$$

Pi(n)=Pi(n−1)+ [(x/′fpix(n)] where Pi(n−1) for n=1 is the numerator for pi at the level of compression under consideration. So for Pi4, the pi we are used to dealing with, Pi(1)=+4+4/fpix(n) for the first solution and Pi(1)+4/fpix(n) for the second solution. By focusing on a quantum state, fpix; AuT is able to look beyond fractals to the bits which give rise to the universe for the first time.

For efficient fusion, the process is reversed.

The relative size of the circles and spirals and other features in FIG. 1 to each other and to the quantum elements which they affect can be important to achieve fractal equivalence and even variations in those relative sizes can be used to target the transitions although on large scales some like curvature might be sufficiently similar given the fpix underlying basis that this is less critical.

### A. Overview of the AI Problem

AI is unable to work efficiently with scientific data. The current scientific landscape suffers from inconsistent data terminology, different languages and fragmented data structures across disciplines (physics, chemistry, biology, engineering and to some extent within those disciplines) hindering knowledge extraction and analysis.

Fractal AI FIP provides for substituting different languages with a single underlying mathematics.

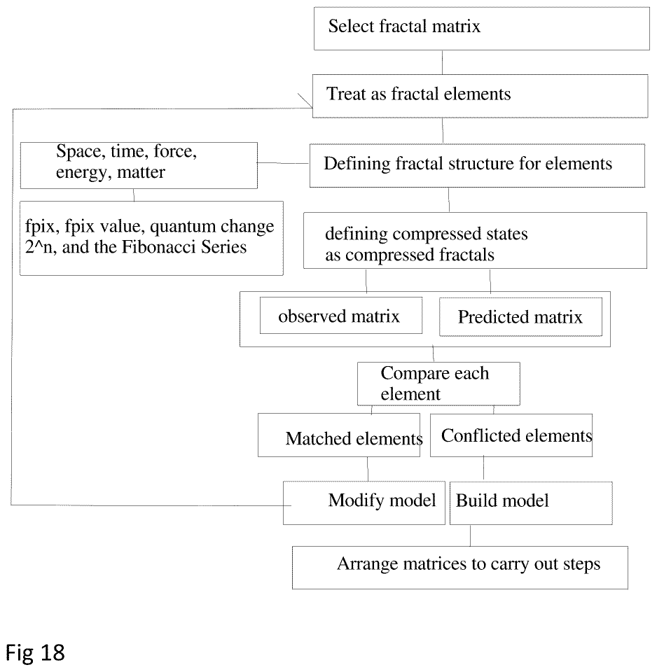

FIG. 18 shows a simplified outline of the process Fractal math is the application of iterated equations. The premise it that foundationally the universe arises from iterated equations. Two clear examples of iterated equations seen ubiquitously are (1) the equation fpix, developed to generate the denominator of pi and (2) MI (Fibonacci) which is seen in the golden ratio which appears at levels above the neutron. Applying mathematics in place of less specific modeling simplifies information storage and analysis. Because fpix can be represented as a bit (moving between positive and negative solutions) it is an efficient foundation since bits are the language used by computers to process information.

Simplification: Fractal modeling is used to simplify the data by breaking the science from articles into fractal components and detect errors and consistencies by comparing the fractal results, simplifying and validating scientific data by converting as much of the word salad making up scientific articles into mathematics “based on common iterated equations” so they can be defined as different fractal representations of a common mathematics whether force, energy or matter based. Fractal modeling reduces the number of variables by converting disparate data types (e.g. matter and energy) into features of a common fractal system, simplifying complex datasets. Resulting non-fractal anomalies stand out, highlighting potential errors increasing clarity and confidence in their interpretations and reducing “hallucinations,” since anomalies become evident. The process is applying a consistent mathematical framework across any or all scientific disciplines by focusing on the math underpinning science.

### Fractal-Based Prioritization:

Repeated fractal features within complex datasets are used as signposts. These features guide the prioritization of relevant data, streamlining the analysis process. Irrelevant data is filtered out or processed at a lower priority speeding up the AI. The AI applications can be used to filter out static/data blocking/other interference as non-fractal compliant, and it can be used to establish and break coding. For fusion this would be to take each fusion reactor, the setup of the fusion reactor and convert everything to fractal modeling to test the fractal model against the results and how far they fall from fractal modeling. Non-fractal descriptors are not based on specific iterated equations at the quantum, gravitational level.

A broad benefit will be trustworthy, effective, and deployable Artificial Intelligence (AI) technologies interpreting scientific data.

The method can be defined using the following steps:
1. Use FIP algorithms to convert physics data to fractals.
   a. convert electrochemical and electronics data to fractals,
2. Use FIP algorithms that can convert the chemical data (from articles) to fractals.
3. Use the fractal origin of constants [gravity example, calculated results compared to observed results is used to gauge accuracy] so they can be used to evaluate the converted data;
4. Using standard algorithms to pull out individual words or phrases which can be converted into fractal elements of chemical data can be seen as a step, and this is traditional in AI parsing of written and spoken words and the form of the information is only exemplary since this applies to information available in any form;
5. It may be appropriate to fractal or non-fractal based algorithms use algorithms to deal with uncategorized information, converting it to fractals (i.e. base iterated equations that can interface) or categorize it as non-technical data;
6. Using algorithms to store data based on common FIP features of fractal data.
7. Using FIP based algorithms to weigh and predict from the data.

a. Existing concepts in algorithms can be used to compare actual results, predicted results and those required by FIP modeling and weigh the data.
8. Using programming to incorporate the results into MI. Each individual element to be identified, categorized, measured and compared between articles.

The following independent steps are relevant to the process:
a. convert chemical data (from articles) to fractals, convert physics data to fractals; it would be more accurate to separate these conceptually based on dimensional transitions,
b. convert electrochemical and electronics data to fractals,
c. convert uncategorized information to fractals (i.e. base iterated equations that can interface).
d. define fractal origin of constants [gravity example, calculated results compared to observed results is used to gauge accuracy];
e. Define the Relationships of the first 4, one example already performed is showing that the relationship of gravity to change in dimension structure at neutron is related to the equation 2f(n)\hat{ }2(n) with a transition from fpix to MI architecture at the neutron for f(n).
f. Store data based on common fractal data.
g. Compare predicted results to those set out in the data in question to weigh the data.

The resulting fractal components and the matrix defined by the content of the article can be rated according to the consistency of the article with the model or based on the ability to fully convert the elements into fractal components.

The process will include modifications in sampling, conversion and in the underlying modeling used for conversion, storage and models for analyzing and grading the converted data. Based on the feedback, the AI system updates its training data, model parameters, and algorithms (Model adjustment via Continuous Learning).

Quantum change means that interactions between matrices are not functions of a complex set (energy, magnetism, force, etc.) over an undescribed concept of time, but instead mathematical transitions from positive to negative values over quantum changes of n=n+1. While this does not change the importance of time-based analysis, it allows for categorization based on quantum changes leading to time.

Interactions are viewed as pretime (energy/force) and post-time (denser information states, electrons and larger and also pre-time elements that are not changing over time). The model should allow each individual element to be identified, categorized, measured and compared between data in the form of articles on the science in question up to a point, some approximation is necessary.

Automation is based on the existing fractal framework compared to known outcomes with the modeling refined to improve the results, in short, a feedback loop. An AI feedback loop is an iterative process where an AI model's decisions and outputs are continuously collected and used to enhance or retrain the same model, resulting in continuous learning, development, and model improvement. In practice, there are at least two types of AI feedback loops: Positive: Users, in this case LLMs, provide positive feedback when the model generates accurate outcomes aligned with their expectations.

Negative: Users report flaws when the model produces inaccurate results, leading to system improvements.

More efficient storage and prediction using this modified data using existing LLM models is possible through reducing the parameters. This includes the ability to automate breaking data into fractal components. The ability to weigh the articles and the ability to weigh the fractal model to grade and/or improve different aspects is a separate criterion. The ability to provide common reference points for associating, comparing and/or storing data based on fractal components.

Data Storage and Sorting with Fractal features

The process is using this base fractal architecture to define or design the coordinates for any purpose.

Creating a subset of fractal dimensions which are used in conjunction with the coordinates of the larger informational framework, similarities (and inaccuracies) can be identified. Searches can target one state, one particular category of fractal transition or compression as well as transitions and collections.

### Embedding

Currently there are at least 15,000 dimensions for categorizing and cross referencing locations of information in some AI applications. We can reduce categories in terms of fractal relationships since a fewer number of features can be used to associate information in technical applications.

1) Similarity Searches can be based on an approximation locator based on common fractal elements, such as neutron related features being tied to different levels of neutron compression.

The process is defining AI data based on Coordinates based primarily/even solely on fractal qualities, this can include using fractal modeling to define the sliding window to embrace information on either side of “rag memory” related to area of association.

Fractal features can help define the features of the sliding window based on common fractal features as well as proximate location.

Results can be Graded based on expectations versus the actual results and then the process can be marked with the grade which grades can be assessed for patterns to improve the process.

Retriever and large language models (llm) get data from vector embedding;

associations connecting coordinates separated by distances defined by dimensions.

Fractal modeling provides a homogenization directed parameter which can be used to reduce the complexity of scientific data, a parameter which is clear as to “how they work, when they fail, and what they are even capable of due to their emergent properties.”

Optimization by Developing AI algorithms that predict and enhance the efficiency of carbon capture processes beginning with translating articles covering climate related content.

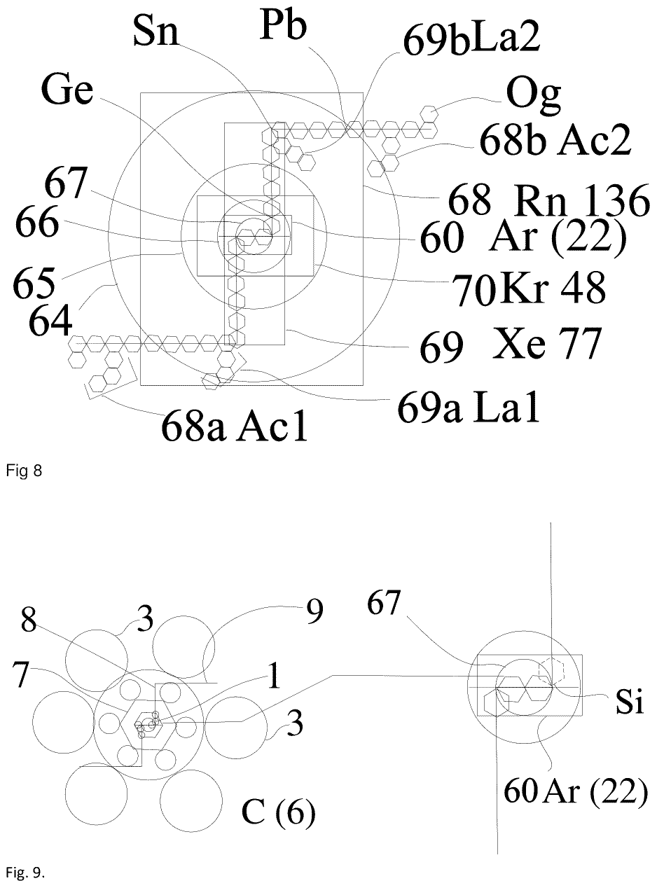

Example 1: The periodic table of the elements can have elements defined by a system of balanced fractals beginning with the proton and electrons as dual fpix spirals (2*1(He), −3(C), 5(Ne),−7 (Si),9(Ar)). In fractal terms, pi can be defined by the iterated equation: pi=sum(n=1 to infinity)4/fpix(x) where fpix= [symbol] −1 [symbol] \hat{ }x+2×* [symbol] −1 [symbol] \hat{ }(x−1). N does not go to infinity, but since it is a very high number at the level where we exist, infinity is a useful estimate. The can be described using neutrons as dual MI (Fibonacci) modeling as shown in the discussion of FIG. 8. In fractal terms: MI(n)−(mi(n−1)+mi(n−2) where MI (1)=0 and MI (2)=1. Reconciliation of lower level proton and neutron counts can also be seen as a function of a ratio of one fractal in a series to the next (1:2, 2:3, 3:5 for example) supporting the fractal relationship between stepped compression states. This defines a dimensional transition from a curvature-based model (more specifically an fpix model and a MI (Fibonacci series) model, at the neutron. Two times MI does not give the neutron count, but if you overlap 2 MI spirals with 2\hat{ }n diameter circles and match the inner diameter circle to the 2/3 overlap you get these lengths: 2(He), 5.5(C), 9.8(Ne),14.1(Si), 22.8(Ar) for the lengths of the spirals within the successively larger resulting circles.

By their nature iterated equations provide a framework to nest increasingly compressed forms of information defined as ct1 states which are solutions to the equation fpix.

Space is a dimensional effect. If you track evolving solutions to fpix with a common quantum change result folds from a one-dimensional linear set of solutions to a partially folded two dimensional graph. This does not mean that the model is completely worked out, it just provides a foundation for understanding why this can work based on observed findings. Existing Components of the fractal model include: 1) Fpix, ubiquitously observed in curvature, 2) MI, observed above the level of the neutron and compression of fpix and MI based on 2f(n)\hat{ }(2\hat{ }n) where f(n) for electrons and protons is a function of fpix and a function of MI for neutron counts. For f(x)=fpix, where n=4, f(n)=7 and 2\hat{ }n=16. This allows the equation of 2f(n)\hat{ }16 to be viewed as 2 sets of 7 units doubling 16 times.

Example 2: Carbon (12) is defined fractally as C1,R1ct4(2), R2ct4(4), R3ct4t17(6), R4ct4t13(6), SA1-4. This defines carbon 12 as having 6 neutrons, 2 inner neutron backbone, 4 in the second backbone layer, 6 protons, 6 electrons and 4 shared areas for bonding. Each of these elements is based on either MI (in the case of the neutron backbone) or fpix(all other parameters) along with the 2\hat{ }n portion of compression equation 2f(n)\hat{ }(2\hat{ }n).

Atoms do not look like the resulting drawings. Galactic equivalents show the collapsed core to be small compared to the larger area “occupied” but the exponentially smaller lower ct states (electrons, photons, pre-photons, the components of space, but fractal equivalents allow for representations showing the relative effective size. Fractal relationships are dimensional features arising from the iterated equations. The “force” associated with bonds corresponds to sharing areas in the drawings which is a function of pi(fpix) and 2\hat{ }compression of information, not the size of the components. A fractal description for carbon can be expressed as: FIG. 7 shows the Fractal modeling of carbon for this purpose by way of example.

C1, R1ct4(2),R2ct4(4), R3ct4t17(6), R4ct4t13(6), SA1-4. This defines the neutron as being in radius 1 (R1) and radius 2 (r2); the protons as being in Radius 3 (r3) as the 17th transitional state leading up to the neutron, and the electrons being in radius 4 and being the 13th transitional state with room for up to 4 shared areas (SA1-4) in outer radius R4(radius 4) for bonding with Oxygen in CO2. A hexagon (hex) in place of one of the 2\hat{ }n circles highlights the geometery bethind observed 6-sided effects.

The idea of a neutron backbone is not presented by the prior art, but compression along fractal lines (where the next solution builds on the prior solution by definition) suggests a backbone of neutrons, surrounded by a proton core which in turn is within an electron shell. The ability to “predict” the neutron count using overlapping MI spirals shows the fractal math to be applicable.

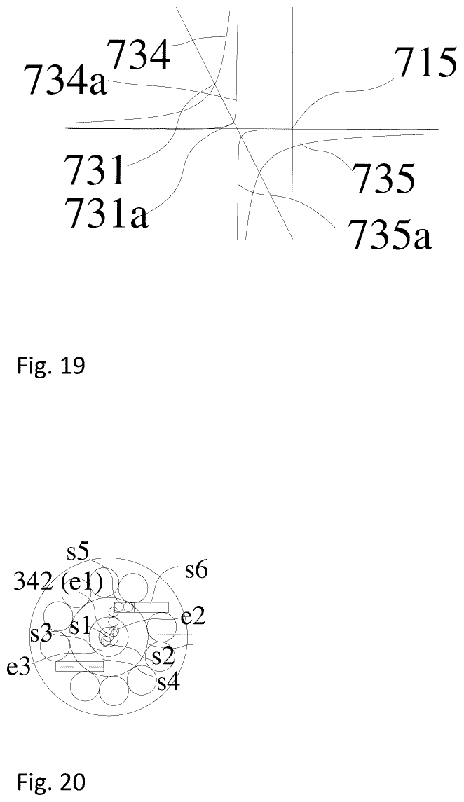

FIG. 19 shows Carbon 31, Oxygen 32 and CO2 as a combination using fractal modeling

A fractal description for carbon can be expressed as:

C1, R1ct4(2), R2ct4(4), R3ct4t17(6), R4ct4t13(6), SA1-4.

This defines the neutron as being in radius 1 (R1) and radius 2 (r2); the protons as being in Radius 3 (r3) as the 17th transitional state leading up to the neutron, and the electrons being in radius 4 and being the 13th transitional state with room for up to 4 shared areas (SA1-4) in R4(radius 4) for bonding with Oxygen which would have a similar designation.

The two “shared” areas (33) defined by the proton area are where information overlaps, fractal representations of dynamic bonding and the “areas (33)” are force equivalents.

Since the information content remains the same the information lost when SA areas are filled is where heat from a reaction comes from allowing energy transitions to be modeled based on pretime change in information which is lost or gained.

While this C1 designation for carbon appears more complicated, it allows the primary isotope of carbon to be described with only 2 dimensional features, ct state (compression state) and radius (R) level. This serves as a starting point for fractal modeling in AI applications for the first two lines of the PTE.

The two areas (33) where information overlaps are fractal representations of a more dynamic process and the “areas” are fractal equivalents of spacing that is not restricted to the locations shown. Since the information content remains the same this “missing area” is where heat from a reaction comes from allowing energy transitions to be modeled fractally based on information which is lost or gained in combining atoms.

For the elements in question, those within R1 and R2 have MI based geometries, those in R3 and R4 have fpix(curvature based) geometries.

Based on Mass consideration, the electron may be viewed as a ct4t13 and the proton as a ct4t16, understanding that there is a dimensional transition between the proton and neutron which accounts for the difference in appearance and mass. Using FIP, a spiral galaxy can be compared to an atom.

FIP modeling of carbon circle 7 shown as a hexagon to reflect observed 6 sided effects.

The golden ratio view of spiral galaxies is documented, the ability to calculate the neutron count using overlapping MI spirals within 2\hat{ }n circles is a relatively trivial math exercise using trigonometry and the first circle equal to the 2/3 overlap. The spiral galaxy reflects an averaging of overlapping MI spirals and fpix based curvature. At the center, the space between the two golden ratio curves, is a black hole core which follows MI architecture. A dimensional transition is reflected by overlap of the information at the galactic center. Viewed from the next lower dimension, this is the “black hole core” of the galaxy. This is what happens when protons transition to neutrons at an exponentially lower scale.

### 2. Categorization

Using fractals with data science to modify AI software to maximize the accessibility.

organization, and value of empirical data. Dealing with the complex variations such as what appears essentially infinite variation in life requires AI and AI requires a base structure, underlying algorithms which can tie everything together to avoid chaos. We can cross-characterize existing, empirical non-fractal data within sub-atomic, atomic, chemical, and biological data with fractal data required by the “AuT” Fractal modeling used to define any features al any level of dimensional existence based on fractals effects within the matrices under consideration.

Categorization is a broad way of describing design by breaking into fractal elements one or more of the following: chemistry (reaction; fuel; carbon capture; rare earth combinations) based on fractal chemical and molecular structure of the at least one matrix; biology (DNA, ATP, biological design, and function); physics including titanic event prediction and control; quantum gravity and time, black holes, dark energy, wave particle duality, etc.); Energy capture, generation, and storage; fission; Quantum Computing using the non-pretime change and pretime changing with non-pretime changing or amount of pretime change to deliver or withhold information; Concentrating pretime elements; qubit function and design utilizing key fractal elements (KFE). CT states are an example of KFE as is quantum change which with the CT states creates pretime change for relative work.

Fractal modeling “integrate(s) multidisciplinary” information because the same fractal model applies to all dimensional features of the universe and even pre-dimensional features which energize the universe.

AuT includes treating AuT fpix changes as plus and minus. There are multiple ways in the electronic spectrum where the plus or minus result can be sampled or applied with fuse length, compression vs decompression; abs vs spew; pretime and 11 post time change. The absolute definition of change at ct1 is a quantum count common to all points in the universe, “absolute time” or more accurately “absolute change” or “AuT dimensional change.”

This allows energy, even time, to be viewed as just another fractal change within a quantum fractal system. Despite exponential complexity at different compression levels, fractal means there are equivalents at every level. Larger atoms mimic galaxies in structure a concept which is the result of a mathematical analysis, not theory.

All of the different manifestations of change and those that result in energy changes can be treated as fractal matrix changes reflecting average pretime change.

The work includes: 1) identifying the fractal patterns which should be associated with the data and (2) looking for fractal patterns at all levels (energy, pre-energy, structural, component, environment, etc.) (3) processing at least by categorization the data based on fractal modeling. In chemistry fractal components and reactivity elements have been identified based on confirmed, but unrefined fractal mathematical features. These features are the baseline fractal structure which can tie everything together to avoid chaos.

### Energy and Radiation Viewed in the Context

Force in general and Energy in particular are represented as the transitions between different states, broadly defined by 2f(n)\hat{ }(2\hat{ }n) transitions where f(n) changes between fpix and MI. Forces arise from these transitions, confirmed by comparisons to observations and explanations of otherwise unexplained phenomena, such as time, magnetic attraction and the relatively weak force of gravity.

Gravity is folding of individual solutions to fpix, called ct1 states, folded into ct2 based on the equation 2f(n)\hat{ }2(n) for n=1 and f(n)=fpix(1, −3,5,7). Carbon's electron shell and proton count reflect 2*f(pix), where n=4. At neutron bonding, the geometry shifts, so in the case of atoms they can be defined below the level of neutrons based on fpix, and above the level of neutrons by the neutron backbone defined by overlapping MI spirals.

Force can be defined as interactions between matrices of different “compression” states as observed from the standpoint of time. Time and its relationship to energy in various forms can be seen as a form of stop frame animation for compression states at and below the level of photons, those below the level of photons being impossible to observe using electromagnetics because they occur below the level of photons.

Magnetism can be modeled as the net transition of “pre-photons” occurring pre-time as viewed from a post time perspective, acting much as a low-pressure system. Waves reflect pre-time changes in position as viewed from the perspective of time. At any quantum moment the photon is the particle of “wave-particle” duality.

For modeling, force is defined as interactions in quantum terms between matrices of different “compression” states (ct is the nomenclature used). Time and its relationship to energy in various forms can be seen as a form of stop frame animation for compression states at and below the level of photons, those below the level of photons being impossible to observe using electromagnetics because they occur below the level of photons. Magnetism can be modeled as the net transition occurring pre-time as viewed from a post time perspective. Waves reflect pre-time changes in position as viewed from the perspective of time. At any quantum moment the location of the photon is frozen, the particle of “wave-particle” duality.

In this model, bonding forces come from shared information and energy results from the release of this shared information based on specific iterated equations and bonding or un-bonding results from inherent features in the underlying equation.

In a single amp there are 6.24×10\hat{ }8 electron flow per second. Under this analysis there would be a flow of 6.24×10\hat{ }8*14 photons, and each photon would have pre-photons (ct4t12 states) which in turn would have pre-pre photon states (ct4t11) and so on.

While this remains approximately the same over long periods of what we call change, the ratio does change because at any point in the universe the transition from net compression to decompression (and for the universe as a whole) changes over time. And time can be viewed in more absolute terms as quantum change in a quantum count. As a system accelerates relative to its localized baseline, the amount of pretime change slows relative to the amount of post time change reflecting the movement relative to the baseline in place of change within the system slowing down the feature of time. Moreover, at the electron level, waves can be viewed as particles. (photons) being in multiple places at once from the perspective of time (vs. being localized for any quantum change at a specific point) so that the ability to do work (energy) can be viewed as the ability to take a pretime change and use it to move things around in a post time perspective. Energy can be viewed as the availability of pretime change within a system.

Putting this all together, one can begin to model plasma based on information states where the number of photons (172) and pre-photons (171) and smaller information states (ct states) increase so that that the separation 58 between the positron 34 of the proton 176 and the electron 12 is “dissolved” in a sea of pre-photons and photons. The “flow” of the pre-photons is the magnetic field that helps maintain the plasma and the amount of change in the plasma is the amount of pretime change in the matrix reflected at the level of the photons 172. The flow also gives rise to the wave forms observed.

We see “waves” as the movement of photons at pre-time dimensional scales as waves generated by “Archimedes wheels” (top left). The more change “pretime,” the more compressed the wave form and the more energy it represents. However, at any quantum moment the location of the photon is frozen. The frozen photon is the particle of “wave-particle” duality.

A “dead photon” with no energy would have no pretime change. A giga-hertz photon (1billion or 1×10\hat{ }9 would have change centered down 8 levels (since this is in base (2*7), 8 levels down is in the range of a billion in base 10.) Accepting for the sake of discussion that the electron is a ct4t13 state, 8 levels down would be ct4t5 states. In a single amp there are 6.24×10\hat{ }8 electron flow per second. Under this analysis there would be a flow of 6.24×10\hat{ }8*14 photons, and each photon would have pre-photons (ct4t12 states) which in turn would have pre-pre photon states (ct4t11) and so on.

To introduce “energy” into the analysis, we must get power from these photons. By defining time in terms of the amount of change below the level of a photon over an observable number of quantum changes, we can treat Time as a dimensional, “stop frame animation” effect that arises between the photon and electron as the smaller particles (ct4t12 and smaller in the example) change position below the level of the photon.

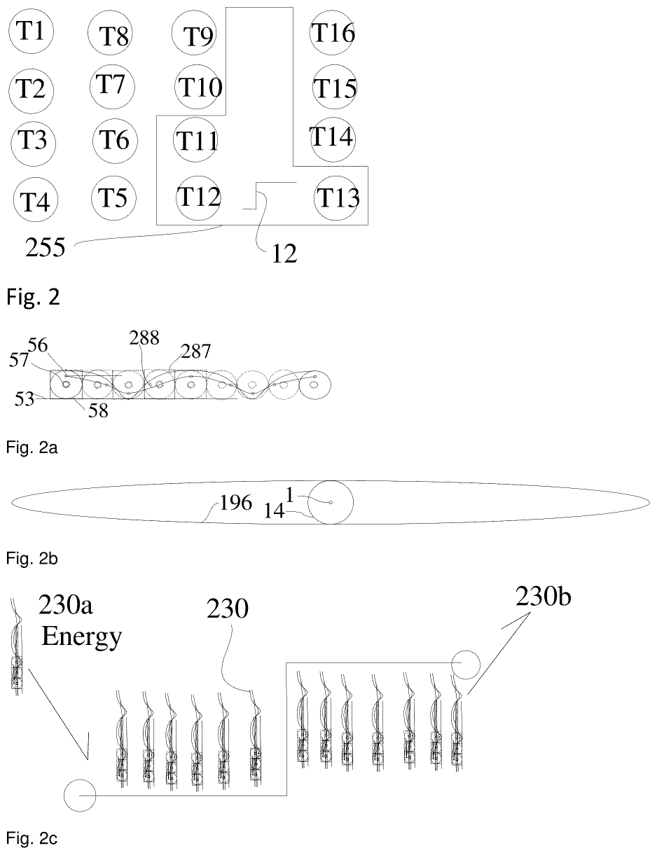

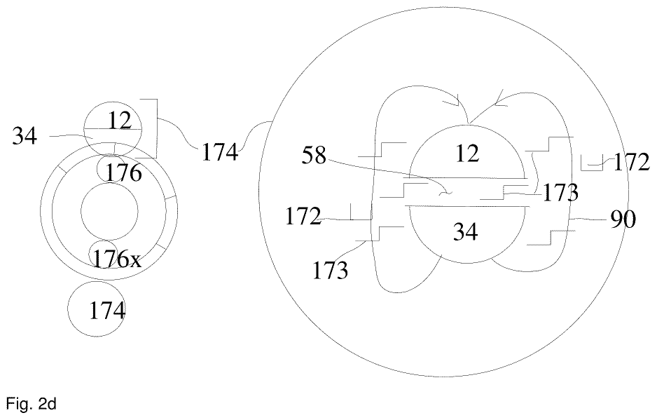

FIGS. 2 through 2d show a Fractal View of Energy

FIG. 2a shows how a photon (or pre-photon), in multiple places at once from the standpoint of time appears as a wave generated by pre-time movement of “Archimedes wheels” (top left). The more change “pretime,” the more compressed the wave form and the more energy it represents.

FIG. 2b shows how scales change in base 14 from 1 to 14 to 196 going from level 1 to level 3. The circle for 196 is flattened so it fits in the space provided.

In this scale a photon (ct4t13 196) is 1/14th of an electron (top right), the pre-photon element (ct4t12 14) is 1/14th of a photon and represents the electric component, the pre-pre-photon (ct4t11 1) is responsible for the magnetic component.

It's worth noting that there is a soup of these particles and of this soup, at the pre-pre-pre photon (ct4t10) there are 2744 ct4t10 states reflected from this soup in a single photon, others around it).

Time and pretime diverge at the photon, so that “energy” represents pretime change at the level of the photon. FIG. 2c shows the electron composed of photons. Each photon 230 is the wave (top left) form of a particle changing before the dimensional effect we view as time. The designation of the bound electron as R4ct4t14 refers to the Fourteen ct4t13 states shown above making a single ct4t14 electron.

FIG. 2d shows the resulting effect of Charge as the exchange in the direction conceptually shown (spew lines 90) of the ct4t13 photons (173) between electron and positron 14 extending from a proton 176. We talk of unbound electrons as “electrons” and they are one half of the “bound electron,” 174. The electron alone is 7 ct4t13 states of the total of 14 ct4t13 states in the bound electron. The circulation of pre-photons around the photon when viewed from the standpoint of time create the effect of magnetism and functioning like a hurricane-low pressure system which is a fractal equivalent.

In this case relativity is the relative amount of pretime to post time change in a matrix. The ability to do work (energy) can be viewed as the ability to take a pretime change and use it to move things around in a post time perspective. Heat is “trapped” within molecular bonds of the matrix, while high energy particles (HEP) are particles with high amounts of pretime change.

### Fission

Referring to FIG. 1, different features of fractal modeling available including layout of overlapping spirals to go with the filtering and collection systems otherwise following fractal equivalents along with transfer PN layers of varying types to charge varying carriers and focusing on positrons as holes which can be manipulated and arranged to increase flow efficiencies in any solar or thermocouple type transfer and opposing charge areas and the like.

The right semiconductor and KFE structure (physical and chemical) can break down or build up to a desired energy and potential and provide for effective producing a solution of photons, positrons and electrons with focused or enriched pretime change from dissolved bonds of atoms and molecules creating a fluid matrix from an overly fluid and overly solid features.

Direct transitions between fission and electricity focus on low wavelength thermocouples. The alternative we address will focus on high energy particles by developing a lead (Pb), tin (Sn) or lead/tin hybrid semiconductor (solar cell equivalents) in conjunction with other SC (FI, C, Si, Ge and hybrid semi or partially functional conductors). The key advance is FIP where energy is based on iterated equations, it can be stepped up or down incrementally based on stepped photon and pre-photon elements. Under FIP atomic structure is fractal giving unique insights concerning the operation of semi-conductors. Radioactive sources generate extremes of energy which cannot be easily translated into energy. Light wave energy particles from high energy particles, can be obtained and used with existing solar cell design.

The universe is fractal. This means dimensional features repeat at different scales, a giga-energy photon and a photon responsible for heat vary based on “stepped,” mathematically “iterated,” features that repeat at different scales. High energy particles in radioactive materials can be separated using the same techniques as solar cells using fractally equivalent atomic elements, where Lead and Tin share sufficient equivalence to other semiconductors to act in conjunction with P-N doping to separate potential.

N is potassium, P is Antimony, doping. Between them is junction of.

Under fractal design this set of discrete steps downward be handled with fractal modeling.

What makes fractal modeling effective is that it allows us to see the “discrete” sequential steps involved in the creation of energy and atomic structures. Cold FISSION, SC (Semi-conductor) design for transitioning directly from radiation to electricity. While using fractal equivalence along with FIP modeling as a process that involves all dimensional manipulation, one of the best examples is the ability to define fission and fusion processes.

It is possible to begin with scaling small scale processes using larger scale fractal equivalents, modified according to dimensional transitions and fractal compression. An example would be using boiling water and showing how it is fractally equivalent to the operation of a thermocouple, solar range energy (or higher) phenomena along with 22 showing the break down and reorganizing energy based on filtering, transportation and application of larger materials. The modeling can be done in either direction given the process of utilizing fractal modeling at scales and at fractally equivalent scales and transitions. Fractal equivalence means that modeling can be based on fractal mathematics directly by translating observed effects at different scales or mathematically defined transitions even at scales which cannot be observed, converting them to FIP elements and then using those results model related processes, all processes being related by fpix. This applies to breaking down fission elements into usable photonic wavelenghts and targeting fractal features of energy, potential, transitions, and atomic structure at the best locations in the matrix.

The invention is defining a process using these transitions. For example, the radiation transformation can be viewed as a landslide of ice moving outward from the radioactive source. This ice must be broken up and melted (separated into the frequency to be accumulated) using filters and heat so they can be collected in a dam in the form here of a semiconductor with N type doping to hold the photons in question at the pretime change rates selected, they are then dropped over a gravity gradient provided by the change in the semiconductor to the P type doped layer while turning the generator atoms in the PN layer so that a potential exists just as it does by turning a generator. This modeling allows for each of those elements to be viewed in terms of fractal equivalents at any scale.

It might even make sense to spin these to create a centrifugal direction for the movement of the pretime change, although the movement of the high energy particles from one or more radioactive core can create the necessary effects with the required geometries which are not a factor of traditional views.

This process can be used to take megawatt features of radiation, break them down in terms of pretime change, concentrating pretime change at the desired levels, absorbing in a semiconductor type material at the desired level, transfer at a PN layer externally.

The semiconductor (SC) locations where pretime change is concentrated and drawn off are based on the need to capture the energy states in a matrix, semiconductor most commonly, where they can be transferred to an N type layer than using the natural flow of a P type layer or its equivalent to a PN layer where it can be drawn off in contrast to them being absorbed by the shielding atoms using KFE to break down or concentrate the desired wavelengths as efficiently as possible which is why lead, with its large structural spirals has too much room, absorbing pretime change as heat (pretime change absorbed into the atomic structure).

Since this happens in bulk, having round (or other shape defined by the fractal structural transitions set out herein) and solid (less room for adding information) atom layers with embedded fractal matrices whose features take advantage of the fractal structural transitions in the Pb or Sn semiconductor layers to separate pretime change, using donor elements to take the higher pretime elements and add lower pretime change to them at the appropriate photon level so they are in the light range and then proximately placed absorbing and transmitting semiconductors (solar P: N type so that absorption for the property pretime level by the N layer creates a potential at the PN interface.

Circular structures (using 2\hat{ }n and fpix resonance architecture in their design and nesting layout) also includes half circles (cups) to capture information as can, as shown in FIG. 1, the combination of these. In terms of pre-photons, these can be captured until they fill “cups” the size of the pretime change needed for the photons to be capture to create this effect even though these would be virtual and controlled from more compressed electron based non-virtual arrangements from our perspective.

Fractal layouts and material which encourages the type of binding and release that increases or designs the type of information release which is critical to the direct conversion to electricity or for whatever effect is possible since the specifics are defined by these fractals. The potential is the area between the circles which is a target location.

If you are looking for photon equivalents or greater than photon absorbing, TI and Bi on either side of the Pb semiconductor or B and N doping with Carbon is possible.

The silicon in the solar cell absorbs photons (light particles). When these photons hit the silicon, they knock electrons loose. These free electrons then flow through the silicon to the n-layer typically doped with elements like phosphorus or arsenic to increase electrons. Holes are the empty places left by the electron, in FIP, the A-positron, positron type electron halves extending from protons, more easily modeled than holes.

While the n-layer is crucial for providing a pathway for these freed electrons to move, the absorption and the initial excitement of electrons happen throughout the semiconductor material is because of a solution of photons in FIP.

FIG. 1 can be used to show how fractal elements can be applied for the different features necessary to get a more direct transition from radiation to electricity focusing on fractal atomic resonance and pretime change at different levels for modeling energy to allow transitions in energy so that, fo example, it can be recombined by taking the fractal elements which have pretime change and rearranging them, then gradually drawing these off, something we already do with sunlight and doped (PN doping) semiconductors.

Elements of fractal transition can be modeled and assembled based on FIP modeling. This applies equally to chemistry and fusion and electronics.

These are the controlling inflection points for the dimensional universe. Targeting these fractal transitions and fulrums (points of resonance) is part of the invention, r efficiently manipulating all dimensional features.

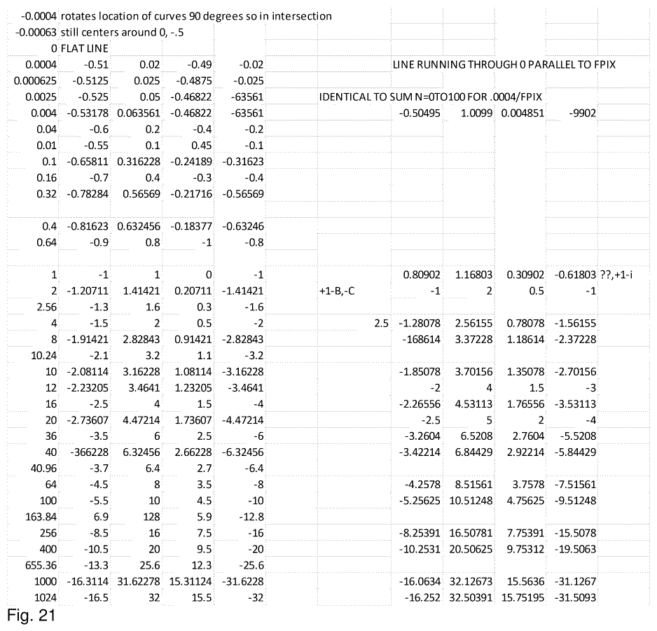

FIG. 21 viewed with FIG. 1 and FIG. 19 charts mathematically the origin of circular dimension fulcrums as points of resonance as a function of fpix. At scale this matches the alignment of the two arms of a spiral galaxy in terms of spacing about a fulcrum, the galactic center corresponding to −0.5.

The features are all part of FIP modeling and as such are all equivalents and evolve along lines which can be defined by FIP in general and fpix in particular. 4 to the n is like 2\hat{ }n and It remains possible that resonance (4\hat{ }n) is a primary source of compression. It is also likely that they work together because the sum(Otox) for fpix also has resonance at 2 (−1,2 for the y positive and 0.5, −1 the y negative). The sum also has resonance at 12 and 20 but it is not the fractal equivalence seen for 4\hat{ }xfpix/fpix. Whether this can work in place of 2\hat{ }n for overall compression can be verified by applying 4\hat{ }n to get similar results for force and the like.

1) Resonance charting 730 in FIG. 1, FIG. 19 and FIG. 2 as a reflection of Periodicity (fpix: 1/fpix) base 4 and the resulting rule of 4's observed in nature. The calculation for the force of gravity suggests that the 1,2,3,4 fpix transition applies. The rule of 4(s) based on numerator of pi: 1, 2, 4, 8 is possible because (1) skipping 3 explains the lack of forces between gravity and those in the electromagnetic spectrum and atomic and there is no resonance at 3. 8 works well in the transition from circle to three dimension, these calculations are shown.

This allows a negative count to play a role as one or more of the building steps.
a. Fpix along with inherent folding, the resulting combinations of information (compression/decompression) and dimension building. Given the existence of n=n+1, n−1, MI and the two iterated equations making up fpix as well as 2\hat{ }n compression and dimension building into circles and spheres from fpix and resonance of fpix, the “single parameter” functions within a limited group of evolving or interacting parameters defined dimensionally as those from the group listed necessary for design.

2) 2\hat{ }n compression, potential between these steps including a focus on using changing areas exposed by 2\hat{ }n diameter circles and spheres; numerator increasing by 2\hat{ }n (1:2, 2:4).
a. Spheres first 1, then 2, then 4 in size going in to out or out to in layering to break up energy, made of lead, possibly Sn (Si, for ex) bubbles. Spheres in the form of bubbles within or without one another and matrices based on the materials features relative to the fractal transitions, particularly electromagnetic and particle features to be separated or dispersed, channeled or absorbed can be used in this fashion. This includes Increasing or decreasing the surface area of interaction, but along 2\hat{ }n fpix based lines.

3) Transitions defined structurally or using different compression features, such as atomic or molecular structure based on the FIP modeling and construction and the resulting process.
a. Shifting geometries from fpix(curvature) based to MI (Fibonacci based) at neutron bonding and from two dimension to 3 where the Basel problem suggests they exist and
b. Shifting base numbering with increasing compression of information state or decreasing compression of information state and 3) shifting overlap from being offset to being overlapping at the same location
c. Coming together vs separation (new) of MI/fpix
d. Compression and decompression in stages for 3
e. Shifting Geometries from fpix to MI at Neutron Bonding: The method involves shifting geometries from fpix(curvature) based to MI (Fibonacci) based during neutron bonding. For fusion, for example, the inflection point transition from fpix to MI can be targeted. In working with pi, the constraints imposed by the more accurate math related to changing dimensions and the Basel problem and the emergent nature of resulting three-dimensional geometries set out in more detail herein.
f. Shifting Base Numbering with Information State Compression including shifting base numbering while compressing or decompressing information states. As information states become more compressed, the base numbering system must adapt accordingly, ensuring efficient representation.
g. Overlapping Shifts from Offset to Same Location: The method involves shifting overlaps from being offset to being directly overlapping at the same location using FIP to maintain consistency and ensuring smooth transformations.
h. Coming Together vs. Separation of MI/Fpix: The concept explores the interplay between coming together (integration) and separation (disintegration) of MI (Fibonacci) and fpix(curvature) elements including balance between integration and separation controlled to avoid instability or loss of coherence.
i. Compression and Decompression Stages for Fractals: The method includes compressing and decompressing fractals in stages. Limitation: Each compression or decompression stage can be viewed as reversible, preserving essential information and minimizing distortion.
j. Predynamic and Dynamic Scaling of MI/Fpix Structures: The process dynamically scales MI (Fibonacci) and fpix(curvature) structures. Scaling factors should adapt to the specific context, avoiding excessive distortion or loss of detail.

The expanding accordion like expansion with positive and negative transitions while increasing the total separation along with the resonance as the numerator of the inverse of fpix shown for graphing fpix solutions and graphing the fpix inverse shown in item 730 in FIG. 1 show evolution of the base dimensional features which can be targeted for prediction and modeling. Fission modeling can start at the radiation source 711, which can be any shape including spiral shaped in one layer, in the concentration design giving rise to fission and any dimension to focus the transitions along FIP lines using the modeling techniques set out here.

FIG. 19 shows the addition of 734a reflecting a plot of the sum from 0 to 100 of 4.00004/fpix and 735a reflection the negative y of that alternative plot where 734 is the positive y for 16/fpix and 735 is the negative seven for 16/fpix to show how these appear relative to one another.

Each layer can be spherical or flattened into a circle to reflect the two dimensional nature of pre-neutron modeling, different layers with different levels of purity and with doping (using fractal design of both the doping materials and how they are included, are ways of modeling of the layers within one another to define desired fractal transitions.

All fractal features are based on the FIP specifics set out herein and while terms like shape, purity, different radioactive and non-radioactive elements are terms of the prior art, but reducing all features and goals to FIP elements allows energy a new, mathematical as opposed to intuitive approach.

Simple layering, as opposed to layer with purity for radiation shaping is shown based on 2\hat{ }n in item 711b and as MI modeling in item 711a.

The order of layers, the concentrations and percentages can be calculated with some specificity depending on the source of energy using similar concepts to traditional energy exchanges but modified to recognize the nature of the transitions as fractal and based on the equations for pre-atomic and atomic fractals. For this reason, we might have multiple layers of lead SC 712 and the locations of the P—N layers (or fractal equivalents) as well as their separation may be modified to reflect the nature of the energy transitions and the high potential changes. For the sake of simplification, the layers are presented showing 2\hat{ }n transitions and in the order of concentration inherent in their construction. There is the Pb (lead) layer(s) 712 which can have multiple 2\hat{ }n, fpix or MI based transitions, each having semiconductor placement at different places within the layer(s) and the same for the Tin (Sn) semiconductor 713, the Ge SC 714, and likely others as the energy is broken down, concentrated and/or focused and separated within the framework. Unspecified layers 715, reflective in that they have strongly filled fractal constructions or absorbing like lead or perhaps silicon might be involved in this layout.

There are semiconductor buffers 716 which reflects the ability to absorb different layers of energy, thereby decreasing the levels with, along with offset SC 717, also absorbing energy at specified levels so that the chamber formed by the perpendicular SC(s) 718 (the exact angle(s) would reflect the FIP modeling and, in this case 1×SC 721 forming a chamber (the number of items 718 on all sides is likely, to reflect and absorb energy (CT states of various energies) and allow for the separation and concentration maximized by FIP modeling.

The depth and width of the semiconductors within and between layers is reflected by 3×SC 719 and 2×SC 720 which take the filtered radiation and continue the process, forming potential separation layers between them to maximize the yield.

Channeling occurs through the interchange of CT states inherent in all matrices and for this reason, given the modeling for overlapping spirals, there are overhanging SC elements 722 envisioned as well as having them offset. Similarly, density of materials 13 and structures can be used to form funnels 723. This is another example of how the concentration of materials can make the net effects better for the results. In the case of items 2,4 and 8 in item 711, for example, these might reflect changing concentrations of U235 so that the higher energy producing results may be external (higher 235 concentrations between 4 and 8) of internal (higher concentrations within item 2 or variations, such as pockets of concentration to control the direction (like the funneling) and quantity of CT states for this purpose.

While the example of 711b is used here, using 711a or fpix modeling can be used. In the case of fpix modeling the negative.

Reflective SC 724 also embody the idea of more radiation absorbing/reacting material to build CT state creation at different locations within the matrix.

Offset SC layers 725 reflect a broader separation concept. This applies to where potential may be drawn off as well as to take advantage of the relative positions of CT states circulation of and within the various elements addressed.

There may be absorption of spew of information based on the alignment of one or paired spiral arrangements 732, exemplary being silicon P or N layers aligned at MI based spirals of varying sizes and here sown as overlapping spiral layout 726 of item 711 to absorb information coming off the 2\hat{ }n circles or spheres or directly from the radiation source.

2\hat{ }n nesting 728 is series as opposed to the nesting internally 711a and 711b and the semiconductor material as nested 2\hat{ }n squares in FIG. 1.

Resonance 730 is shown as dimension arises based on the center of an x/y axis 738 of the initial two-dimensional framework. Fpix resonance 730 results from the 0.5 offset of fpix solution line 732 and the resulting inverse (1/fpix) association 733 which results in a positive 7 curve 734, negative 7 curve 735 and the resonance top 736 and resonance bottom 737 which results from the intersection of line 732 with curves 734 and 735 which is later reflected in the overlapping spiral form.

The nature of folding as being incomplete ensures separations at the quantum level reflected at higher levels of compression. The universe doesn't collapse because fractals resulting from the underlying universe do not collapse all the way as they fold (FIGS. 17, 1 and 19 show this). The compression of information occurs according to the increasing number over fpix initially, the MI so that the space between the arms of galaxies and atoms is filled with information folding along those lines which leave these successively larger spaces between them according to the underlying iterated equations and the “observed forces” merely reflect this folding and unfolding and averages.

There are stepped, particularly by 4, 16, 64, compression of resonance 730 locations, there is also compression (and decompression in all cases where compression is used) along the curved lines to the points of intersection and the points where the two halves of the solution approach. These features can be used to model and achieve desired dimensional transitions under fractal modeling.

Unlike traditional Semi-conductors or thermocouples, the potential is exponentially greater giving rise to higher voltages, but also making the transitions higher than are otherwise available. The energy in many cases is more concentrated around the high energy particles, which is the energy in heavy particles, representing the pretime change of the states within those high energy particles, is not only high, but concentrated and bound so that it must be drawn out. Having FIP designed bubbles, in fractally relevant materials, within the various layers can focus pretime change from individual particles. Whether these create a metal foam, a semiconductor foam or a foam of one material inside another, this is another way of approaching t. transition of the information into a usable wavelength where heavier particles are involved and the same modeling shown for item 711 can be copied for this purpose around or within the various P—N layers for providing absorbing information states and to change those into the usable form for the absorbing layer.

FIG. 21 is a Chart showing Resonance and the rule of 4(s) which can be used to target electromagnetic, chemistry, fission, and fusion modeling. In base 10, fpix 7 intersects at multiples of four for the numerator as 4\hat{ }n/fpix(1/fpix,4, 16,64,251), item 730 in FIG. 1. Because any multiple of 4 is your numerator, this regular pattern corresponds to intervals or oscillations in the curved function. an equation is linear, and the reciprocal of the equation is curved. At fixed values of the numerator x, x\hat{ }2, x\hat{ }3 the result is an integer. Otherwise, fpix is largely an irregular fraction. Since observed curvature is based on 4(and 4\hat{ }x is the series of resonance points for fpix and its inverse) and since the radius of a circle yields 2\hat{ }n as it doubles, the observed feature of 2\hat{ }n is seen to be mathematically resonant and tied to doubling circles because pi is defined at a point of resonance (it almost had to be since the universal appearance of curvature is necessary evidence of the applicability of fpix as a quantum bit) and resulting circles reflect 2\hat{ }n compression.

Other resonance is shown, as at 0.04, 0.16, at 1 and 0.01, 2.56 (compare 256), 100 and 400; all relevant, in base 10 the consistent symmetry is 4\hat{ }x observed at dimensional scales we are most familiar with pi(4) based on 4 as the numerator over the sum of fpix being pi.

This informs the nature of fpix. Changing the numerator to a negative number does not show the symmetry since while the curves remain the same for inverse of fpix, they are rotated 90 degrees to the point −0.5,0 so they no longer intercept at the fpix line through −26.5 and instead the lines of the two curves converge on either side of the fpix line along both the x and y axis center at −0.5,0.

The line representing the sum of fpix value, as opposed to those values alone, moves left as the number goes up so at −100 to 100 it is at approximately −100.50001, 00002); at 0 to 100 it is at approximately 50.50002 to 0(0.00004). This is just a chart of various representations of resonance, fpix, x/fpix(where resonance with fpix can be seen); sum(a to b) of x/fpix, for example which can be graphed to observe resonance. The intersections at powers of x=4 highlight underlying symmetries, where certain multipliers align more perfectly between the linear transformations and their reciprocals. These points of intersection can be viewed as resonances-harmonic mathematical inflection points which can be viewed from the perspective of time to include frequencies where the transformations synchronize up, yielding integer values (note that because of the −0.5 offset, the x.5 values are integers which start at −0.5). Resonance, at its core, is about synchronization and amplification. In many systems, it occurs when the natural frequency of the system matches the frequency of an external force, leading to a significant increase in amplitude.

Exemplary applications of fpix Resonance include Mechanical Resonance: At the natural point of resonance, the natural frequency, force is maximized, potential is at its greatest.

Acoustic Resonance: Musical instruments, like guitars, use resonance to amplify sound.

Electrical Resonance: Circuits can resonate at certain frequencies, enhancing signals within those frequencies, used in radio tuners and filters.

Mathematical Significance: resonance indicates specific points where the linear and reciprocal functions align in harmony, leading to integer results. These harmonics or resonances tell us about the inherent properties and symmetries of the functions.

Resonance leads to large-scale effects from small inputs, amplifying signals or creating harmonics that can be harnessed in practical applications and applied for deeper understanding of systems.

Core Dependency, which is the behavior and characteristics of the entire system are influenced by how the foundational equation and its reciprocal interact. Resonance points would function as stable states or key attractors within the system.

Resonance is a factor in locating transitions and Predictable Patterns: At specific values (x\hat{ }n, noting fractional and −x\hat{ }n features set out) the system exhibits predictable, stable behavior due to the resonance. These points signify crucial states or transitions that the system tends to gravitate toward, hence the strength of the rule of 4 in a nature defined by fpix and this resonance inherent in compression.

Harmonic Alignment is represented by the integer results at these resonance points function as harmonics, creating predictable intervals that define the overall system's structure and dynamics.

Fractal stepping means that Subsystem Coherence built on this foundational equation modeling aligns with the resonance points. By disrupting targeted aspects of a subsystem applications the system's harmony can be disrupted, causing irregular behavior. Chaotic features are eliminated in place of expected fractal transitions which can be used to model returns to harmony or moves away from harmonic resonance. Iterative Relationships means that the output of one step becomes the input for the next. This iterative process ensures that each step maintains alignment with the core resonances, preserving systemic coherence and providing targets of transition based on the stepped approach in either direction.

In mechanical or acoustic systems, resonance frequencies define the stable states. For example, in a bridge, certain frequencies can induce resonance, leading to amplified vibrations. Better designs can come from targeting the multiple resonances at different compression levels.

Because they reflect underlying fractal applications, economic foundational equations defining market behavior can be improved using resonant points to signify stable economic states or equilibrium points.

In Electronic, Chemical, and biological systems, resonance indicates stable states or homeostasis points where the system is in balance.

The resonance points function as anchors, creating stability and predictability within the system. When aligned properly, the system operates harmoniously; otherwise, it can exhibit irregular behavior.

For fpix, the x coordinate increases by −1, the y coordinate increases by 4(−1:4 slope) for the portion above y=0, below y=0 the transition 2\hat{ }n, −1*2\hat{ }n gets very small in pir\hat{ }2, the lines become a cross closing in on 0.5 (point 5) forming a cross as opposed to offset lines. As you grow from infinitely small to infinitely large you get a bigger separation. If you use one thousand as the diameter, the left foot (along the x axis) for green, separates at the same rate as the y axis. Both lines eventually end up on zero (for all intents and purposes) and form a cross about the 0.5× axis with an increasingly small separation. The base four analysis can be varied by looking at sums over different amounts of information. For 4*r\hat{ }2 as r changes in increments of 10 (e.g., 10,1, 0.1, 0.01) the intersection of the left bend with y changes as a function of 4(e.g. 40,4,0.4,0.04, etc.). If 3r\hat{ }2, it does the same thing, but 30,3,0.3, etc.) but always about 0.5 which reflects the 2×transition (1/fpix means 0.5) between points in the modeling.

Hence you have a y axis intersection that drops by 4*radius\hat{ }2 while the lines converge on NEGATIVE 0.5,0 the bottom running y always slightly more than −0.5, the upper running y always slightly less than −0.5. Fpix brings the lines together based on an intersection based on the numerator towards the x axis based on base 10 and closes in as set out in the spreadsheet.

Proton to Neutron Fusion can be designed as transitional dimensionally. To get fusion the positron has to be balanced with a collapsed electron cloud and then pushed into the t15 mix along with balancing with other proton-electron pairs; the “subduction” of the t12 state of an electron layer into the proton layer at the positron 34 which would shift with the location of subduction. Stabilizing plasma is contraindicated. Instead of containing fusion, the process is to focus on (1) compression substitutions to combine states that tend to stay compressed, which includes providing enough information of the right type at the right sequence and location so that stable structures can form, (2) collapsing about resonance based fulcrums for proton-positron-electron pairs and (3) providing an environment for alignment and combinations using the f-series pairs shown and confining and balancing these with the fpix series 1:1(2), 3:3(6) and 9:9(18) pairing of protons and (4) fractal alignment on either side of a fulcrum of lower information states and (5) collapse around the fulcrum of the Neutrons with (6) balancing the sharing of information as absorption and spew.

Neutron backbones form a foundation for proton cores which stabilize the neutrons by providing information necessary for the neutron to continually “absorb and spew” information. Proton to Neutron fusion requires ct4t15/ct4t12 folding around a stable ct15 backbone with supporting CT states along overlapping fractal lines absorbing and spewing to maintain the collapse of the ct4t12 states in pairs within paired ct4t15 states. Having “pre-paired” protons and neutrons (hydrogen) ready to build balancing neutron backbone is easy to envision as a step in “cooling neutrons” separated by plasma generation and recombined with electromagnetics pulling the plasma away leaving the neutron backbone halves of whatever size more exposed to one another.

The process includes using exchange of CT state features between at least two matrices to remove non-compressive CT states, increasing compressive absorption in CT states, balancing ct4 states within the neutron backbone with ct4 transitional states, overlapping core ct4t15, balancing absorption and spew at each stage as well as the net release of excess spew in the form of decompression pretime information.

The CT state matrix can be imbued with compression tending KFE features, crudely referred to as being sequentially energized into plasma and de-energized (collapsed in a way that releases pretime change at the level of photons) along KFE mandated fractal lines.

An example of generating plasma around neutrons is with microwaves through a conductor, contacting at the points of the conductor where balancing plasma is desired around an overlapping neutron core; pretime state changes excite the separation of protons and electrons from metals; but this is a process properly tied to information exchange and not non-fractal approximations using terms like energy or electricity even though those terms are measurable.

Multiple and successive streams of CT states of a certain compression state can stabilize one another. Electrons can be concentrated and mixed with protons and then a central neutron core is added as the charged particles are concentrated and accelerated out to stabilize them. Protons are central to the electrons but having them start on the outside might be beneficial as they would pass through the ct4t11 clouds pulling everything together. Likewise, having a negative charge to the neutron tube especially towards the end might help to pull protons towards the neutrons which can be accelerated by having them pulled along by a strong electromagnetic accelerator and the separately accelerated protons (opposite charge) due to the shared absorption from the protons as the EM field is reduced. Protons (P) can also be provided in the form of metallic generated plasma from microwave level energies through conducting wires making contact around a source of neutrons.

Mechanical (shaped as by spiral funneling, like what you see with water through a drain or with a chamber with screw pathways in the center or at the walls or both, possibly offset (oppositely spiraled and expanded to increase mixing), using electromechanical and sub-electromechanical (magnetic ct4t11) means to mimic these forms to concentrate the balanced matrix and organize multiple FIP based plasmas at different levels. Facing plasma streams can replace the physical and virtual chambers in part with an offset to encourage rotational balance at the point where the two streams of information meet, with different plasma streams supplying the CT states for at least some of the “six steps of fusion” including stabilizing CT states or states to balance at each stage the process or encourage the continuation to the next stage. Directed energy” or “plasma ignition” is analogous to concentrating states with high pretime change rates (tending towards gamma rays) from those with less pretime change (tending towards infrared). KFE includes Neutron donors within the plasma distributed to encourage overlapping neutrons with surrounding protons and electrons on either side of stabilizing FIP based plasma fulcrums.

When one thinks of the inverse fpix positive y 734 and negative 7 735 of FIG. 19 coming together about an offset 0, −5 731 one can envision fusion through shifting compression from two offset directions.

If one wants to make efficient fusion, compression according to fpix(as opposed to the steps of MI) occur reflecting how fpix gives rise to a circle, not just by defining the opposing curved surfaces, but the counterpart of a point between the two curves positive y 734 and negative y 735 which expand evenly from successively smaller curves about the offset 731. At very small values of r, the two curves appear to approach a cross centered on x=−0.5, y=0 and as the radius moves outward, the center point stays the same but the separation expands even as the intersection with the y axis increases according to the numerator used times the incremental change, e.g. if the increment is 10 and 4 is the numerator, the increase in the intersection is 40. To target fusion if you are trying to build dimension and bring points together, you can mimic the fission process in reverse, roughly 3 down to 2, then 10, then 2, then 5 and so on until the reaction is complete. Likewise, you can target 0.5 off center where the compression occurs bringing the points together with whatever mechanism is desired to create the desired acceleration.

The nature of iterated equations allows complex structures to be assembled from “units” of base components. The neutron backbone of Argon appears to be built on carbon units of 4 times 5.5 carbons.

Proton counts and electron orbitals are functions of 2 times fpix, namely 1, −3,5,−7. Two times the negatives (−3,−7) give rise to the semi-conductors up to Si), 2 times the positive values (1,5) give the noble gases through Ar.

We see nuclear forces based on 2\hat{ }n in 711b in FIG. 1 and this is reflected, in fractal terms in decompression photographed above right in an exploding galaxy (also seen in an exploding neutron star).

The spiral, un-collapsed effect is seen in spiral galaxies (right), the golden ratio curves (in green and blue) reflecting the net effect of MI overlapping spirals and curved fpix architecture about a relatively small “backbone” of black holes.

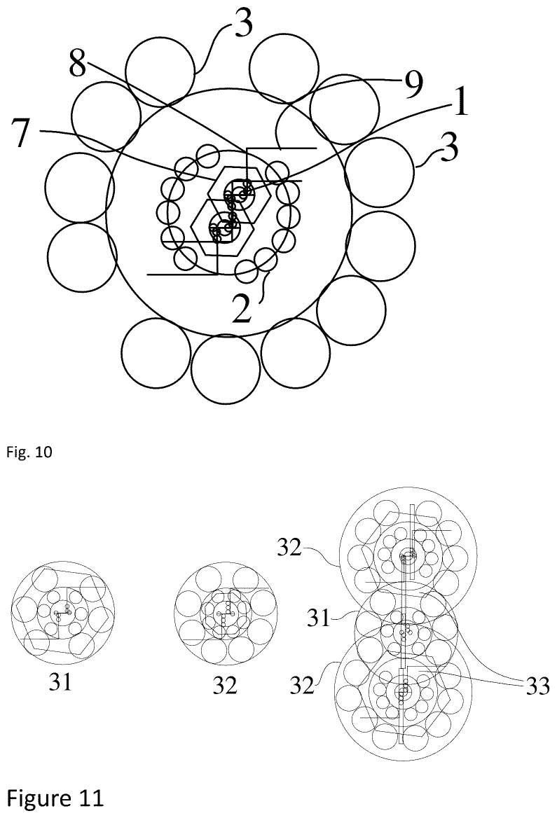

In FIG. 9 Carbon (6) is shown next to Argon (22) with carbon geometry-based neutron units 5.5 units shown as item 7 in carbon, along with associated protons 2 and electrons 3. The circles are 2\hat{ }n circles based on the 2/3 overlap as the diameter of the first circle. The neutrons count is based on overlapping MI spirals and 2\hat{ }n compression. FIG. 10 shows the Argon of FIG. 9 but with the protons and Electrons added. As shown in FIG. 8, At higher neutron counts, the neutron backbone of the atom, is built on “neutron units” averaging 5.5, giving rise to 11 and 12 base backbone pairs, ignoring isotopes. Transitional Argon falls in the middle ground, capable of being defined by a single core MI spiral or a two core MI*5.5 architecture. This is an “inflection point,” a critical element seen in FIP for identifying transitions, including fusion and fission.

Boron/Potassium elements of the P-N doping layers on different columns. There is base 18 (2*9) present in the periodicity (18) of lines 4-7 of the periodic table of the elements. Because of exponential changes in compression, the periodicity of the protons is secondary to the neutrons. Up to Argon, the neutrons bond without pairing into sets of 6 and control the periodicity; past argon, the proton periodicity begins to exert itself as base 18 (2*9), the next step “Backwards” is base 14 (2*−7). The Lanthanides and Actinides drop back to base 14 being free of the primary neutron backbone as spiral arms off of the primary spiral. Note that La and Ag themselves are part of the base architecture, leaving a 14 (2*7) unit proton architecture. Hydrogen in not a true atom as it has no neutron backbone.

Lead falls within the semi-conductor category (Column 14, Tetrels). We can sequentially step down and separate energy from radiation using existing models for transitioning energy, namely: semiconductor chips of layered Pb (Lead), Sn (Tin), Ge (Germanium), Si (Silicon) or C (carbon) doped with P—N type doping to separate positive and negative charge to create current. At the inflection point, at column 3(½ the N base 6), the Lanthanoid (row 6) and the actinoid (row 7) series begin. This reflects the interplay between the neutron backbone and proton core, you have the neutron arms (5N to 3P ratios) coming off on either spiral N arm to provide the base 14 P arms.

At the inflection point, at column 3(½ the N base 6), the Lanthanoid (row 6) and the actinoid (row 7) series begin. This reflects the interplay between the neutron backbone and proton core. The neutrons control the proton count in the first three lines, then it shifts to neutron/base 18 protons and this shifts where the neutron arms branch of at rows 6 and 7 where the reduced neutron concentration allows the protons to revert to base 14 P arms.

The Fibonacci sequence is calculated as follows: N(x)=(n(x−1)+n(x−2) so it involves the addition of the two prior solutions. Fpix=−1\hat{ }x+ (2×*−1\hat{ }(x−1)) and Pi(4) =sum(n=1toz)+4/fpix(x)+4/fpix(x+n) where z is defined by the extent of compression, typically estimated at infinity, but actually on the scale considerably less. What you find is that protons follow a base 18 (2*9) architecture and neutrons follow a MI architecture which reflects the change in dimensional structure at the neutron. Rows 6 and 7 of the PTE have a base 18 (2*9) and base 14 (2*−7) architecture, reflecting multiple base numbering features. Column 3 in rows 6 and 7 are broken into base 14 elements in terms of proton counts. This type of breakdown is typical in fractals with multiple base numbering systems.

The neutrons count is based on overlapping MI spirals and 2\hat{ }n compression. The Fibonacci sequence is calculated as follows: N(x)=(n(x−1)+n(x−2) so it involves the addition of the two prior solutions. Fpix=−1\hat{ }x+ (2×*−1\hat{ }(x−1)) and Pi(4) 2=sum(n=1toz)+4/fpix(x)+4/fpix(x+n) where z is defined by the extent of compression, typically estimated at infinity.

At higher neutron counts, the neutron backbone of the atom, is built on “lengths averaging 5.5, giving rise to 11 and 12 base neutron counts, ignoring isotopes.

Argon falls in the middle ground, capable of being defined by MI*1 (column 1) or MI*5.5 (column 2) architecture. This is an “inflection point,” a critical element seen in FIP for identifying transitions, including fusion and fission. At the inflection point, at column 3 (½ the N base 6), the Lanthanoid (row 6) and the actinoid (row 7) series begin. This reflects the interplay between the neutron backbone and proton core. The neutrons control the proton count in the first three lines, then it shifts to neutron/base 18 protons and this shifts where the neutron arms branch of at rows 6 and 7 of the periodic table where the reduced neutron concentration allows the protons to revert to base 14 P arms.

Following the base 6 pattern, these arms on either side of rows 6 and 7 track the structure of the electron/positron interface providing a balanced structure about the same base 6 architecture (at 3, there is room for another 3 to form a 6) and it is seen that these structural features match the same structures seen at galactic levels. The transitions from quantum changes smooth out rigid structure from the distinct overlapping MI spirals to the graceful curves of the spiral galaxies and atoms.

Since lead has a semiconductor fractal structure along with dense neutron rich arms, it is excellent shielding. This represents the semi-conductor (SC) concept in terms of scale and geometry in a basic conversion system designed around 1) known SC design shown in concept, 2) known Periodicity (reflected in the PTE) and 3) fractal modeling.

The location of the P-N interface and the layering (for example having silicone SC(s) within the lead, tin, or lead/tin/Ga interface)). If you were to look deeply, it might appear that the neutron counts appear off for Lead and Tin, the math is similar for Germanium in terms of the number of sets of 6:5.333Ge vs 11.333Sn vs 20.8333 for lead. For the base 14 elements, these would be less compressed and outside of the orbit of the main atomic structure as a result, also expanded relative to those contraindicated by the mathematical nature of compression. Using the 5/3 rule (the approximate ratio between fpix and MI), that means there are between 11 and 12 neutrons to match each group of 7 (two of these) protons. Since the neutrons are still operating approximately on the 5.5 carbon model, there are two of these on each side counting down 3 units of 6 from the main arm and then another 3 units of 6 (18) on the other, 3 units of 6, really closer to 15(5/3*9), on each of the main spiral arms.

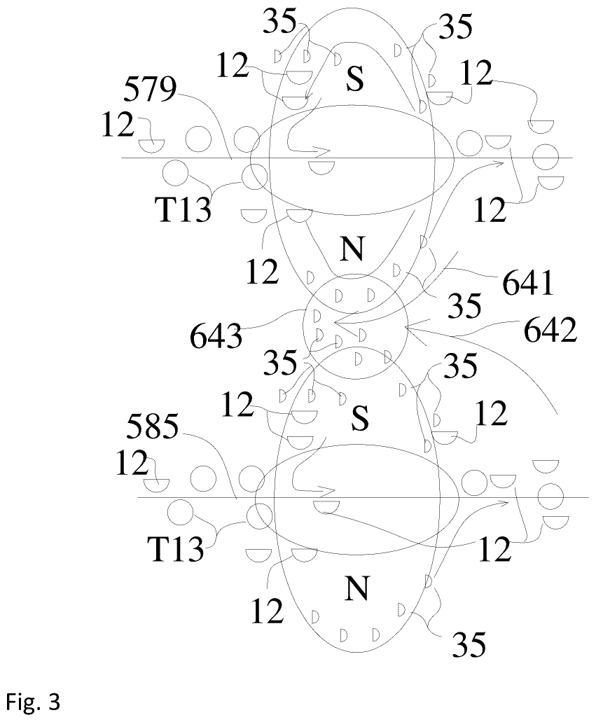

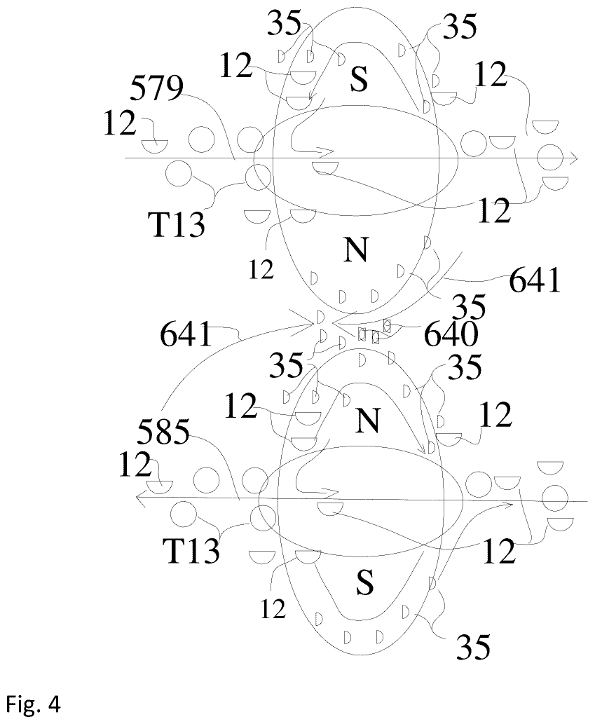

The traditional view of Fission does not consider that all the “energy” in the system abides in “pretime changes” manifested at the photonic level where time becomes apparent as a form of stop frame animation based on these pretime changes. FIGS. 3 and 4 show that all energy is pretime change which allows us to see magnetic repulsion at the post time net effect of pretime dimensional levels. This model can be used to increase the output for an electric motor, a goal for phase 2 along with superior shielding from radiation. These equations represent solutions for f(x) in the iterated equation CTx=2f(x)\hat{ }2\hat{ }x, which leads to folding, force, and other emergent phenomena.

The relative strengths of gravity and strong force allow a check using the compression equation (2f(n)\hat{ }(2\hat{ }n)) and calculating the results.

For MI: Fibonacci (f(n)=1,2,3,5,8,13): For n=2: 2f(n)\hat{ }(2\hat{ }n)=4\hat{ }4=256; For n=5: 2f(n)\hat{ }(2\hat{ }n)=18\hat{ }32=1.47×10\hat{ }40; For fpix(f(n)=1,−3,5,−7,9): For n=1: 2f(n)\hat{ }(2\hat{ }n)=2\hat{ }2=4; For n=4:−14\hat{ }16−2.18×10\hat{ }18 for this step, but the total compression is 21 1.12905E+30; For n−5: 2f(n)\hat{ }(2\hat{ }n)=18\hat{ }32 Neither ratio is precise, but if you use fpix for initial folding element (think boson) to get to fpix(4) and transition to MI for the strong force (n=5,f(n)=8) you get a ratio of 24 1:8.5×10\hat{ }37(3.4*10\hat{ }38/4) which matches the observed force ratio of 10\hat{ }38 for the gravity to strong force ratio. The suggestion is that n=5 up to the proton with a base of 2 *−7, then shift to n=5 with a base of 2*5 for MI modeling at the neutron. This means the 2π electron would be based at least loosely on a base 14 (14 photon units per electron unit).

### Proton and Electron Counts

### Electron Charting

Electron “full orbitals” are 2 times the absolute value of fpix(1,−3,5,−7,9). for fpix values of n=1 to 4. This is two times 1, 3, 5, and 7. The electron count (full shell) matches 2×fpix solutions 2*fpix gives full electron orbitals (2,6, 10,14) just like the Proton count for noble gases reflect 2×fpix solutions, skipping the negatives. 6 The proton counts for noble gases reflect 2 times the positive fpix solutions (namely, 1, 5, 9 for Helium, Neon, and Argon. The next noble gases proton counts are multiples of for Krypton (4×9), Xenon (6 times 9), and Radon (10 times 9, but with 4 less protons showing the importance of the neutron backbone at these scales). These transitions reflect the positive fpix values up to Argon and then build from the negative fpix values.

The initial “noble” change is 1(2 since there are two spirals); then 5(10); then 9(18) skipping every other result in the rush towards noble relationships for the proton count.

The 3(6) and 7(14) remain relevant as semi-conductors, shells filled to give stabilized repeatable, connectable with efficient electron state exchanges; the 3 as the 6 unit/carbon-like ring structure.

In fractal modeling, there are no electron orbitals as such to fill. Electrons are associated with and balance the protons in an atom. Balanced layouts and the other fractal features may be targeted for fusion, fission, chemistry, electromagnetics.

Dissolving this balance between the AuT positiron and electron gives rise to ionization. Electron orbitals are discrete due to discrete ct4t12 or ct4t11 information exchange. Lower states being too small to show up being 14\hat{ }100th smaller than the forces we use. Fpix modeling with N=ct4: If we use f(x) for ct4 is 7, then we have a base compression equation. To get an electron of consequence we can use the ratio of ct4t12: t16 which is 2.6×10\hat{ }-5 meaning the electron is 20.89 ct4t12 states which is robust electron compared to model using 5.4 ct4t12 states (if it were in t13 states it would be 1.5 t13 states). Transitions are base 14 transitions between ct4t12 and ct4t13 and there is no clean transition between 20ct412 states and ct4t13; you already have 29 1.49 t13 states within the ct4t12 states. Such a “two times” 20.89 ct4t12 unit; ct4t13: 41.78/14=2.98 ct4t13 states.

This may be a balanced 1.48:1:48 product compared to a 1.005:1.005. Much of the information is “massless” because it is in the form of energy equivalent pretime states. The pair would absorb ct4t11 (or ct4t12) from a source which would give it a charge type exchange (positive or negative).

The ct2: ct5 ratio for gravity to the strong force works well in this scenario. (1296:1.47×10\hat{ }40=1×10\hat{ }37). If the initial ratio is used, it is 14 or 196:1.47×10\hat{ }40, the 196 number working well). We are working in base 7 or if balance is included, base 14. There are 1.49 T13 states and, multiplying this by 14, there are 20.9 ct4t12 states in base 14 fpix mathematics, 3.84 times as many ct4t12 states as seen with base 10 math.

A pair of electrons under this fpix analysis would be 41.8 ct4t12 states or 3 ct4t13 states less 0.015 lower information states. One might alternatively call it 2 ct4t13 states plus a cloud of almost one ct4t13 state's worth of ct4t12 and lower information states.

Keeping in mind that this is a base 14 system, this lower information represents 7.03 ct4t12 states.

If we look at what happens if we ignore the 0.4, the shadow of lower CT states around the 5 ct4t12 states, instead of 5.44×10\hat{ }-4. This is 5.438806×10\hat{ }−4 in the calculation and is the measured mass of electron divided by the measured mass of the neutron which is complex because much of the information stabilizing the electrons is considered without mass. Using 5×10\hat{ }−4, the resulting measurement (0.0005×14) is 0.007. When you average these two results you get 7.30716×10\hat{ }−3 as the electromagnetic effect, an average between the pure ct4t12 and ct4t12 plus lower state effects. This is 99.8657% the same result using the non-fractal morass of equations used to calculate the fine structure constant (FSC).

The problem with shifting base numbering is that we must derive it from observations consistent with the modeling. Full ct4t12 pairing gets a ct4t13 in base 10 may be cleaner than a pair of t13 states, but we also have the evide. of Helium equivalence to paired t13 states. Full shells appear as t13 pairs in the 1.45:1.45 analysis. The evidence includes the resulting similarity of the electron to the fine structure constant and fpix pairing numbers. This is the process of using the fractal design, categorization, and use of features which we have.

The electron viewed as paired T13 states with clouds of ct4t12 (and lower) can find a fractal counterpart in paired neutrons in helium. Neutron MI spiral folding reflects the overlap where fpix folding gives way to MI overlapping spiral folding yielding solutions in the “calculated” column below. The

If you measure the length of the spirals which have been confined by the Fpix defined circles, the lengths yield the neutron count for the first 3 lines of the PTE. Multiple other features of atoms are apparent, and the F-series ratio of protons to neutrons established are maintained for all subsequent atoms. The initial “noble” change is 1 (2 since there are two spirals); then 5 (10); then 9 (18) skipping every other result in the rush towards noble relationships for the proton count. The 3 (6) and 7 (14) are relevant reflecting semiconductors; the 3 (5) as the 6 unit/carbon-like ring structure.

Visible transition from Fpix to MI in the equation 2f(n)\hat{ }(2\hat{ }x) occurs at the proton to neutron fusion location and is visible in atomic and post atomic structures.

The MI overlapping spirals are reflected in the appearance of MI in large structures from atoms to galaxies as shown with the example above right. The model shows shifting base numbering complicating an already complex compression and decompression (folding and unfolding) mechanism. Fractals give rise to curvature (accepted science, although not with this breakdown of elements) and fractals givegiving rise to the electron shells as well as proton and neutron counts. The measurements of the “S” line lengths in FIG. 20 to inflection points in the drawing on the left gives rise to the neutron count showing the transition from a curvature based universe to an MI based universe. The first leg of the MI spirals overlap 1:2:1 and the circles defining the inflection points represent 2 times increases in the diameter of e1 with a diameter defined by the overlap of the spirals which can be represented by the equation 2\hat{ }n.

| observed | calculated | observed | | |
|---|---|---|---|---|
| neutrons | running total | protons | Element | fpix |
| 2 | 2 | 2 | He | 1 |
| 6 | 5.499093 | 6 | C | 3 |
| 10 | 9.824209 | 10 | Ne | 5 |
| 14 | 14.12121 | 14 | Si | 7 |
| 22 | 22.79091 | 18 | Ar | 11 |

Overlapping spiral inflection points (intersection with 2\hat{ }n circles or change in fpix) yield the resulting atomic/post atomic MI structures and reflect the transition from fpix related structures and MI related structures. The initial overlap is 1:2:1 allowing the remaining numbers to be calculated using simple trigonometry.

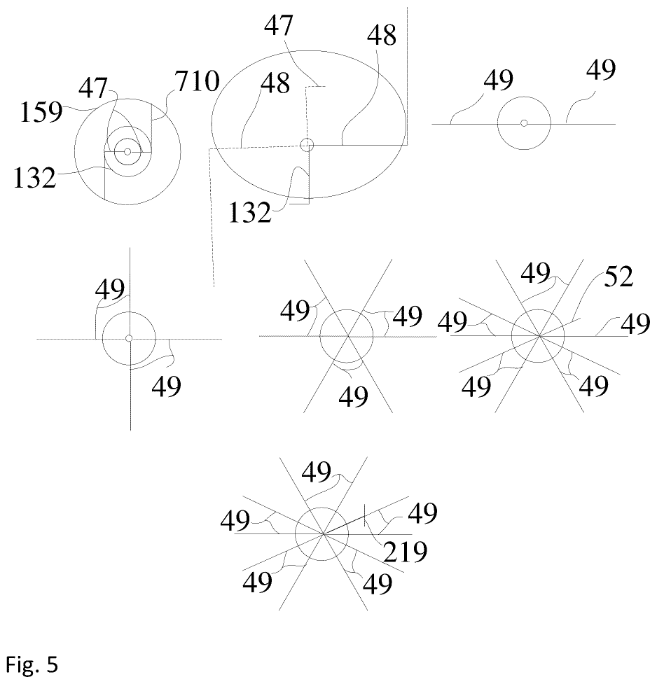

FIG. 5 shows how if we use the equation 2f(n) for f(n)=fpix, we can derive both the proton counts and full electron orbitals.

Force can be derived from folding according to the equation C(x) (compression for x)=2f(n)\hat{ }(2\hat{ }n) and this also allows us to cross-check the fpix to MI transition.

Plasma is just a solution of photons dissolving the α-positron to electron information sharing. The fractal bonding of CO2 (left) is a shared ct4t12 electron area.

The Basel problem, fpix and the transition to curvature and dimension The Euler Basel Solution was Sum (1 to inf)1/(n\hat{ }2)=pi\hat{ }2/6.

The positive and negative values of fpix are not required to calculate pi, but they are the most direct pathway. They can also be used for solving the Basel problem. While going out to infinity is a good theoretical concept, it is unnecessary under FIP because pi is defined based on a finite number of places defined by the solution 2f(n)\hat{ }2\hat{ }n to staged compression.

### The Basel Problem Solution Reimagined Using Fpix:

An example of improved modeling can be seen in the Basel problem,

$$Sum ( 1 toinfin ) 1 / n \hat{} 2 = pi \hat{} 2 / 6 = 4 + sum ( 1 toinfin ) [ [ 4 / fpix ( n ) ] \hat{} 2 ] / 6$$

$$6 * sum ( 1 toinfin ) 1 / n \hat{} 2 = [ sum ( 1 toinfin ) 4 / fpixn ] \hat{} 2 = [ sum ( 1 toinfin ) [ 4 / [ - 1 \hat{} n + ( 2 n ( - 1 ) \hat{} ( n - 1 ) ] ] ] \hat{} 2.$$

Note that while infinity is used as an effective result at high levels of compression, it is a large number, but not infinity.

Note that n\hat{ }2 can never be negative and this means that the alternating value of fpix is irrelevant to closing in on pi but is instead a function of the steady increase in value of the denominator, the smaller reciprocal stepped evenly yields pi.

The negative values of fpix approach the values of 1/n\hat{ }2 faster, but not exponentially faster.

If one eliminates the factor of 6, the corollary is that the ratio of the Basel sum(1/n\hat{ }2) to the sum of pi\hat{ }2 approaches 6, is 6 at infinity. Similarly, the ratio of square root of the Basel sum to the square root of the pi sum approaches the square root of 6, approximately 2.4495.

The relationship between the square root of an infinite sum of 1/n\hat{ }2 to a sum of 1/fpix\hat{ }2 is the square root of 6 (sqr(6)). The reciprocal relationship is that the relationship of the infinite sums of n\hat{ }2 to the sums of fpix is 0.4082 approximately.

As in other cases, we need to adapt the nomenclature for the new mathematics. We use Pi(4) for pi based on a numerator of 4 applied (as shown below) to fpix. Pi(B) or Pi(4/sqr(6)) is the curvature solution for the Basel problem. Note that (sqr(x) used for the square root of x) to give a consistent nomenclature.

The solution to the Basel problem is, for any number of points, being at least two, the last two points averaged based on Basel (x series)= [pi [4/sqr(6)]\hat{ }2 as shown below where this is pi calculated using fpix in the manner shown herein where n=4/sqr(6) which is approximately equal to 1.633, the reciprocal being 0.61237.

Note that if pi were based on the numerator of 4(instead of 4/sqr(6))pi*2/6 (using fpix for the calculation) would approach 6 instead of 1 (equality).

Under this analysis the equation PiR\hat{ }2 becomes R when pi is based on the Basel equivalence of Pi(4/sqr(6)). That is when pi is calculated using pi(4/sqr(6)) as the numerator and fpix for the denominator summed over the appropriate number of points) reflecting an inflection point in dimensional building as at this point pi(4/sqr(6)) for an infinite number of points=R. Since sqr(6) is an irrational number, the answer short of infinity is on either side of the solution.

At one point pi(4/sqr(6)>R and immediately beyond that in terms of curvature based on evolving numerators of pi pi(4/sqr(6)<R.

This inflection point can be used, for example, in computer processing design and function since it is an inflection point observable as the origin of dimensional change from two to three dimensions.

### A Look Beyond the Basel Problem

We showed how fpix: 1/fpix yields observed resonance. The growth of Dimensional Changes can be targeted as a repeated process involved in moving from energy to matter, particularly where it shifts at the level of neutron bonding and the collapse of information from a fpix based numbering to an MI based numbering.

The ratio of pi(x)\hat{ }2 to other values of pi is x\hat{ }2/(x-n)\hat{ }2 so for pi4: pi3 it would be 4\hat{ }2/1\hat{ }2 (16/9); pi2 would be 4\hat{ }2/2\hat{ }2 (4), pi1 (16/1) (4\hat{ }2)/(3\hat{ }2).

For pi(3) the result is that the ratio of Pi(3)\hat{ }2/B would change to 6*9/16. These can be expressed as a fraction 27/8 which has a square root (real number).

For Pi(2) the ratio would be 6*4/16 (6/4−3/2). 3/2 is 1.5 which has a square root (real number). For pi(1) it would be 6/16 (3/8). 3/8 is 0.375 which has a square root (real number).

These are not fanciful relationships. We are looking for and at are reasons why transitions occur in math, transitions leading to compression and to shifts in geometry. 6*sum(1toinfin)1/n\hat{ }2=sum(1toinfin) 4/fpixn=sum(1toinfin)[4/[−1\hat{ }n+(2n (−1)\hat{ }(n−1)]\hat{ }2 When we take out the 4 and 6 (as 4/(sqr6)) we find an equivalence between n\hat{ }2 and −1\hat{ }n+2n(−1)\hat{ }(n−1) which can help explain the origin of 2f(n)[which is fpix originally] and 2\hat{ }n [the inverse of n\hat{ }2]. An equivalence is reached quickly at small values of n.

### Squared Results:

$$Sum ( 1 toinfin ) 1 / n \hat{} 2 = pi \hat{} 2 / 6 = sum ( 1 toinfin ) [ [ 4 / fpix ( n ) ] \hat{} 2 ] / 6$$

$$6 * sum ( 1 toinfin ) 1 / n \hat{} 2 = sum ( 1 toinfin ) 4 / fpixn = sum ( 1 toinfin ) [ 4 / [ - 1 \hat{} n + ( 2 n ( - 1 ) \hat{} ( n - 1 ) ] ] \hat{} 2$$

Cubed and quad results.

Instead of closing in on 1, when 1/n\hat{ }3 is used, f(fpix):f(Basel\hat{ }3) closes in on 1.755 for pi(4/6\hat{ }(1/2))\hat{ }3. This compares to the classical solution closing in on 1.202, but also inexact and not related to pi.

The Role of fpix in the calculation of Phi.

The fpix equation, which represents a series derived from an iterated equation group, plays a crucial role in connecting the Fibonacci sequence and the golden ratio.

$$Φ = 1 - 2 cos ( 3 π / 5 )$$

$$Φ = 1 - 2 cos ( 3 / 5 * ( 4 + 4 / Σ ) )$$

Where Σ represents the summation of the fpix series:

Σ=Σ((−1)\hat{ }x+2×(−1)\hat{ }(x−1)) from x=0 to the applicable compression state value of x.

### Visualizing the Golden Ratio Expressions

The ratio of pi(x)\hat{ }2 to other values of pi might be expressed as x\hat{ }2/ [(x+1)*n]\hat{ }1/2 so for pi4: pi3 it would be 4\hat{ }2/1\hat{ }2 (16/9); pi2 would be 4\hat{ }2/2\hat{ }2 (4), pi1 (4/2) (4\hat{ }1)/(2\hat{ }1) and pi(0)4\hat{ }0/2\hat{ }0=1. These last 2 are indicative of the overlap of 1:2 seen in MI modeling (the overlap is 2 and the extension past the overlap is 1 and where the neutron is sized using 1 and the proton is a multiple of 1.

The relationship between MI is relatively easy, phi (the Fibonacci curve) and Pi is phi=1-2 cos (3pi/5) in circular geometry and MI=lim (as x approaches infinity) MI (x+1)/MI(x) noting that infinities don't exist due to compressive steps never arriving at infinity.

phi, pi, and 5 (a key Fibonacci number) appear together where we see Pi transitioning at a specific point to MI. Phi=1+/−2 cos (3pi/5). 3pi/5 is 12/(5*fpix)=2.4/fpix. The cos3pi/5=sqr(3)/4.

So Phi=1+2sqr(3)/4=1+2 cos (2.4/fpix). (Phi−1)/2=2sqr(3)/4=cos (2.4/fpix).

Pi(2.4)=1.885, approximately 60% of pi observed. The suggestion is that the collapse at the transition between MI and Pi equates to a 60% loss in 2-dimensional space as things transition to 3 dimensions. On the other hand, the actual value of Phi is 1.618 which is roughly 51.5% of pi which suggests a 50% loss of 2-dimensional space. The problem with 51.5% is that we are not comparing the same geometries. In moving from Pi(at pir\hat{ }2 where pi=4 from the chart) to Phi we are seeing a collapse of information at the ratio of 12/5. There is no 12 value of fpix, but there is a repeating value which is seen at different scales which is 2*3-6. If one looks at continuing MI geometry for large atoms, the value of 2*6 is repeating. As is seen in smaller atoms, the negative values of fpix tend to form a base for observed transitions. In fact, pi(12) is 3 times observed pi.

So, at the point where curvature is 3 times the size of curvature observed (OS), the get a collapse by a factor of 5 or the ratio of 3×OS:5

### Chart of Dimensional Changes.

1/fpix converts square to circle, square circumference is 2xr to get the diameter which is equal to a side then x4 for area of square and circle. The same concept applies for area conversion from a cube to sphere which allows the in-between transitions to be viewed based on these changing geometries.

Fpix exists independent of resonance which serves to anchor compression states. We can derive other features of dimension from this base equation which are important to fusion modeling and matter to energy transitions as well as to finding usable photonic and pre-photonic transitions.

The circle and square (2pir and 4×2r): 2pir can be seen as pi(8) (2*4/fpix). 2*r is the diameter of a square. So, the difference between square and circular geometry can be viewed from the perspective of a substitution between diameter of the circle and the (from 4/fpix) for the square. For this reason, the series 1/fpix defines the transition between straight line geometry defined by a square and circular geometry. As pi(x) approaches 1, pi(4/square 6) as determined by the analysis of the Basel problem, the geometry of the circles shifts to the geometry of a square and the reverse is true going to pi(4), Pir\hat{ }2 (area of circle) would equate to Pi(4/1). The defines 2 sides of the square with the series [(2*r)*(2*r)]/fpix. If you remove the series fpix, you have the area of a square.

We can prove this without integration or differentiation. As r approaches zero, this might be seen as approaching pi(2*4)=6.8 using fpix and it would form a baseline as such. If we use pir\hat{ }2 as the baseline, the better solution is pi(sqr(2*4)) or pi(sqr(8))=pi(2.83) approx.=2.22. because if we compare the two, we must look at this as effectively the square root of pir\hat{ }2 as r goes to zero where area and circumference are the same and equal to zero.

### MI Plus Fpix to Get Phi.

Phi (the golden ratio made from the combination of MI and fpix is 1.618 approx.):

$$Phi = lim ( n approaches inf ) MI ( n + 1 ) / MI ( n ) .$$

This can be expressed as Phi=1+/−2 cos (3pi/5)=2 cos (12/(5*fpix)

This contains the transition equation (fpix) allowing curvature from the straight-line equation. pi(4/sqr(6)) using fpix for sum(0 to inf) (1/n\hat{ }(1/2)). This is the Basel number (1.633 approximately).

Observed as 1+/−2 cos (3pi/5) The Taylor series: cos(x)=1−(x\hat{ }2)/2!+(x\hat{ }4)/4!−(x\hat{ }6)/6!

Allows us to describe this in terms of fpix: −1+[12/(5*fpix)]\hat{ }2/1!−[12/(5*fpix)]\hat{ }4/2!+12/(5*fpix)\hat{ }6/3! . . . This can be simplified: −1+sum(1toy) {[(−1\hat{ }(y+1))12/(5*fpix)]\hat{ }(2*y)/y!}.

### Spheres and Cubes Area

4pir\hat{ }2 is the area of a sphere. This equates to (4*r*4*r}/fpix. The area of a cube, on the other hand, is 6*area of any one side or 6*(d\hat{ }2). If you equate the diameter and radius, it is 6*(2r)\hat{ }2=6*4r\hat{ }2. (0.524 approx.).

You have a ratio S16r\hat{ }2/fpix: C24r\hat{ }2.

The difference is the factor S16r\hat{ }2/24fpix=Cr\hat{ }2. The factor resolves itself 8/12, 4/6; 2/3;

so S2r\hat{ }2/3fpix=Cr\hat{ }2 This is the ratio between two results offset by 1/fpix changing to a ratio which is 2/3fpix ratio. As d approaches zero, the area of the surface approaches a common geometry.

### Spheres and Volumes

4/3 pi r\hat{ }3 (more specifically pi(16/3). The sphere volume can be rewritten: (2\hat{ }2\hat{ }2)/3fpix. For the cube, this is r*r*r. The difference is the factor 16/3fpix Pi(16/3) (5.333 approx.)

The volume and area therefore follow the rule generally, but with a factor offset of 16/3fpix and 2/3fpix instead of 1/fpix.

Observed for the volume of a sphere and the volume of the cube.

The factor of 8×between the area and volume calculation can be attributed to a flatten sphere to a 3-dimensional sphere (2\hat{ }2\hat{ }2/3 instead of 2/3).

The concept of being able to arrive at the correct solutions without integration and differentiation (depending on direction) provides modeling for looking at a fourth dimension (ct5).

The suggestion is that the 5 in the MI pi series (pi(12/5)) between pi(12/(sqr(6)) and pi(16/3) is that the MI compression series to get neutron bonding occurs within and independent of the continued 3 dimensional compression of space and with the base (10) (that is 2×5 is 10) that we observe based on the two sets of resulting spirals. This would allow for the observation of compression in base 7(14) and base 9(18) observed in atomic electron orbital and proton counts while allowing a split off in base 5(10) in neutron bonding. The division by 3 allows for the observed overlaps of MI spirals to define neutron structure and can be targeted as such.

Using the formula above:
Pir\hat{ }2 (area of circle) would equate to Pi(4/1) or Pi(12/sqr(9))=pi
4/3 Pir\hat{ }3 (area of sphere) would equate to Pi(16/sqr(9))=(4/3) Pi.

We have (to calculate curvature using fpix) 2\hat{ }2 or pi(4/sqr(6)) for 1/n\hat{ }2 and we have 16/3 or (4\hat{ }2) or (2\hat{ }4)/sqr(9) for fpix with 4/3 pi r\hat{ }3 (more specifically (4*4/fpix))/3) defining the resulting sphere.

When moving from 3 to 4 dimensions there are choices. In this case, the most likely choices (this is not observed as directly) you get to 2\hat{ }6 or 2\hat{ }8 divided by sqr(12) or sqr(3\hat{ }3) for four dimensional change with fpix [pi(2\hat{ }6/sqr12); pi(2\hat{ }6/sqr(27);

pi(2\hat{ }8/sqr(12)); pi(2\hat{ }8/sqr(27)).

In all of these cases pi is calculated the same way summing for the equation which yields pi for 4, expressed here as pi(x)=x+sum(nfrom 1 to y)(1/fpix(n)[noting that at pi(4) y is a large number based on the number of ct1 states necessary to get to the level of compression shown, but not infinity. If we treat MI transitions as independent (side by side) to transitions in Fpix and focus solely on fpix compression, we see that this involves somewhere between 1.13e30, 1.7e70 and 1.4e156; looking at gravity, there is the suggestion that the sum runs between 1.13e30,5.18e27 and 3.4e38.

$$Sum ( 1 toinfin ) 1 / n \hat{} 2 = pi \hat{} 2 / 6 = sum ( 1 toinfin ) [ 4 / fpix ( n ) ] \hat{} 2 ] / 6$$

$$6 * sum ( 1 toinfin ) 1 / n \hat{} 2 = sum ( 1 toinfin ) 4 / fpixn = sum ( 1 toinfin ) [ 4 / [ - 1 \hat{} n + ( 2 n ( - 1 ) \hat{} ( n - 1 ) ] ] \hat{} 2$$

Gamma: The factor for time dilation is G=1/(1−v/c)\hat{ }1/2.

1 ct1 leaving=1 movement; r/c\hat{ }2 reflects the number of ct1 states within T at one point vs T at the next point. This suggests (2\hat{ }1) 2/sqr(3) for 1/n (one dimensional change); and for “zero dimensional change) 2\hat{ }0/sqr(1)=1 (unity) which is suggestive of the accuracy of the modeling. This means that these measures of curvature can be used with compression to look at transitions, including those between primary particles. In looking at the transitions, there are multiple base numbering systems which provide information, in base 6, 6 is 10, and we must be mindful when dealing with multiple base numbering systems.

FIG. 2 shows scaling ct3 to ct4 transitions. T1 represents 10 (base 10) or (base 14) ct3 states marking the first step in the 10-step transition to ct4 states, there represented by t16. T2 would have 10 T1 states (base 10) or 14 t1 states (base 14).

The electron 12 is somewhere between T12 and T13 although it would have a cloud of lower states around it which is the case for each CT state. Time 255 arises between ct4t11 (T11) and the electron based on stop frame animation effects of all the lower states as perceived from the standpoint of a time-based electromagnetic system of the type we experience.

The scales involved are comprehendible as fractal multiples. The transitions build according to an exponential increase whether 2\hat{ }n or a transition between 2\hat{ }n and x\hat{ }n the exponential result for x as a whole number greater than 1 means that there are exponential transitions between CT states. Continuity past ct4 is ensured since ct5 states, black holes are an observed example. Modeling indicates ct5 states are not just the large structures securing galactic spirals, but that they exist within, or at least existed within, spherical moon and planet bodies giving those their 3-dimensional features.

Hydrogen is not “an element” because it does not have a neutron backbone. Heavy versions of hydrogen do not have a neutron backbone, even though neutrons are present. A neutron backbone requires two neutrons share information, be balanced and stabilized by surrounding information states, namely protons and electrons. Modeling suggests that protons are ct4t15 states and a special type of positron which is one half of a ct4t13 state, the other half being the electron. This eliminates energy, space and time in favor of changes to fpix and compression states related to fpix. Going back to the discussion of SE and Bohr, we are “quantizing” the wave form of electrons as 5.4 ct4t12 states (See 5.4/7; 5.4/14 base) where the0.4 reflects the ct4t11 (and lower) CT states in the electron matrix. Varying the number of these pretime CT states (each ct4t11 is a quantum photon element.

FIG. 3 shows how electromagnetic fractal fields appear frozen between quantum changes and how magnetic attraction works. FIG. 4 is the same as FIG. 3 except it shows how magnetic repulsion works. We “see” pretime states as magnetic effects and space.

Time and pretime change are “frozen” in these figures. From a time-based perspective, the electrons pairs shown as t13 states would appear fixed about top wire 579 and bottom wire 585. Due to current through the wire, the movement to the left or right of electrons 12, a wire and being pretime these mechanical changes at ct4t11 gives rise to magnetic effects, the ct4t12 states are the electrical component. Being pretime, we only see the effects of the “magnetic” portion of the energy solution.

In both figures electrons are shown as t13 states T13, which are made up of t states as electrons 12. Circulating around the electrons 12 are M+states 35 which are the magnetic ct4t11 equivalent of electrons which M+states are shown rotated at 90 degrees and half size reflecting their exponentially information content (approximately 1/10th that of an election) and their offset orientation which leads to the elliptical appearance of the cloud of information surrounding the top wire 579 and bottom wire 585. The amount of separation from the wire to the circulating M+states magnetic field reflects the extent of the magnetic field portion of the current.

The M+states rotate around the T12 states pretime and their positions are seen as waves as a result. If we take time out of the mix we can see that the two cooperating circulations in FIG. 3 make it relatively easy for the M+states from the bottom wire 585 and top wire 579 to mix, exchange and the result, comparable to high and low pressure systems at scale is to draw them together. This is reflected in the area of sharing 643 In FIG. 4, since the path of rotation is opposite for the two flows of M+states between the top and bottom wires there are collisions 640 which lead to a buildup of these states which, from a post time perspective is seen as magnetic repulsion. The pretime change inherent in the M+states are what powers the ct4t12 states as they not only circulate but exchange information. Because the ct4t11 states are pretime we only see the net effects as repulsive. Our ability to get post time “work” from pretime “change” is the energy effect.

Several factors affect the wave pattern, the distance from the wires of the flow of information which may be increased and disturbed by coiling the wires as is known in the art of generating magnetic fields. Another factor is the amount of pretime change in the M+states which can be thought of as the number of revolutions about the t12 states over any period of time. Increasing the speed of rotation, the movement pretime, results in a shorter wavelength. The more the wheel changes within a given distance, the shorter the wavelength; hence the more “pretime” changes, the shorter the wavelength and the more energy in a ct4t11-12 matrix.

The figure shows frequency generation from a particle moving along a pretime length creating a wave effect when viewed from the standpoint of time and where “energy” is the ability to use this pretime change in a post time change environment to do work and to provide, from the perspective of time “increased space” or “increased pretime change” viewed as energy within the framework on the left expanding the apparent space occupied by smaller particles when viewed from the standpoint of time.

Energy is pretime change; frequency is energy reflected as the number of pretime changes in ct4t11 rotation about ct4t12 seen from the viewpoint of time; Planck's constant is the size of the ct4t11 states which are subsequently viewed as energy from the perspective of time in rough terms.

Plasma and plasma control can be defined by the mix of fractal information of a particular type within a defined matrix much like an airplane can be buffeted from a time-based perspective by changing winds.

Knowing these circulating structures exist they can be used to move and empower transitions in higher CT states.

FIG. 5 shows proton charting with balance based on fpix and balanced solutions. The fpix solutions are 1,−3,5,−7,9,−11. These are the only solutions applicable.

The 2 inner arms each have a value of 1, then there is the negative value of −3, and for the next arm and so on.

The Fractal origin of Proton and Electron Counts shown graphically based on 2f(x) where f(x)=fpix reflecting the balancing (the factor of 2) critical to the structures defined; 2*1=2, 2 *-3=−6, 2*5=10, etc. The “even” results give rise to the noble gases, the “odd” results define the semi-conductors.

Referring to FIG. 5, there are the two balancing hydrogens 47 for a helium atom 132 here identified by the effective radius of the proton core. Also shown are the 3-length negative value arms 710. There are two of these waiting to be filled to give Carbon which area is identified by the potential carbon core radius 159. Next is Neon which has 5-unit balancing arms 48. Items 47 and 132 are shown for reference. Next is Argon where the scale has been changed. The negative value arms −7 are not shown, but the sum (14) would yield the next semiconductor, Silicone. For Argon there are two balanced arms 49 giving the 18 proton total observed. After Argong, there are 4 9 unit arms 49 for Krypton, 6-9 unit arms 49 for Xenon and for Radon there are 9, 9-unit arms plus a broken arm which is 5 unit unbalanced arm 52. While shown this way for clarity, balance suggests that the broken arm and one of the 9 unit arms 49 would each be 7-units and balanced, although different from the 9 unit arms. Each of these 7-unit arms (not shown) might come off of one of the 9 unit arms, by way of example.

The repeating “9's” indicates that the repeating feature of the neutron backbone is the that shown for Argon (2×11=22) as opposed to the balanced “5's” of Neon although radon indicates it might be the balanced 5's with a bridge of 2×1=2 between the two neutron backbones.

Also shown as an example of larger proton balancing is Uranium which is shown with 10 9-unit arms 49 and at the place where the unbalanced arm 52 would have ended there is a helium proton pair 219. The relevance of this modeling will be discussed in more detail.

The broken Radon may correspond fractally to the electron-proton interface.

### Electromagnetics: Category Specific Science

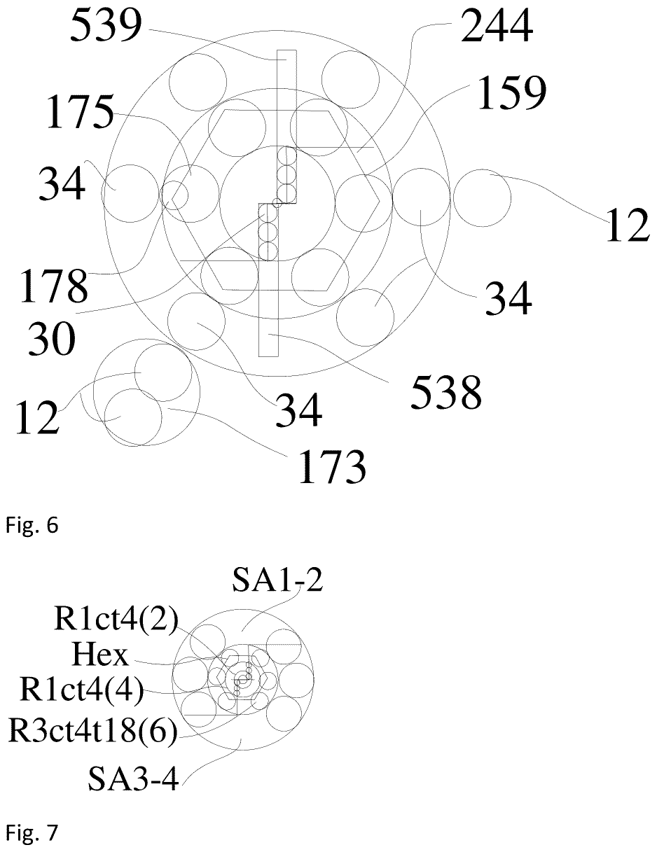

FIG. 6 shows how atomic structure allows for exposure of outer “electron proton pairs” allowing the electron-positron bonds of an atom to be extended along fractal lines through electron pairing which provides stabilizing fulcrums on either side with positrons on either side of the protons paired for circulation of CT states and balance. As shown in FIG. 6 there is a copy engine 539 and bottom engine 538 each of which contain 3 neutrons 30. The spirals that define the location of the six neutrons and this carbon equivalent are not numbered for clarity as this information is covered in previous patents. Outside of the circle defining the location of the neutrons is a second circle which defines the six sided structure 244 of the balancing protons 175 within each of the protons is an unbalanced T15 state 178 which allows the sharing of information with a positron 34. Each proton 175 has a corresponding positron 34 the positrons 34 are balanced with either an electron 12 or by an electron pair represented by a T13 state 173.

This shows eight 6-sided structures 244 about a common neutron fulcrum 644. This forms a total of 48 neutrons identifying it as the neutron core of the primary isotope of Krypton. This shows how the spiral model defining the netron core has begun to collapse showing curved instead of linear spiral form but based on the underlying 6-sided fractal form.

One looks at Xe with 77 Neutrons and the backbone model is called into question. In order to understand why this is not a problem, we look to the measured overlapping spirals as set forth in the table above which lays out the math for overlapping spirals which yields for carbon, the six sided structures shown with FIG. 6, being 5.5. When one uses this math result and divides the 77 observed neutron one gets a balanced six sided structures, the 8 six sided structures for Krypton plus the fractally significant 6 more. This shows that the math, as opposed to the visual manifestations, is controlling and continues to provide repeatable, fractal results.

Radon presents a special case. While the neutron count of 135 is a close approximation of the average between 5.5 and 6 (138 Neutron average); Radon also fails to follow the property proton count. However, fractal math suggests a combination.

The 4 less protons may be relevant. When the neutron count has 4 removed, it represents a balanced 22 (136−4=132) 6 unit states. The suggestion is that the extra neutrons are balanced by being trapped in the neutron backbone and there remains a balanced 22 six-neutron unit atom with the requisite number of protons (86) to balance it. While hardly dispositive, this matches also with the combined 8 six-neutron unit Krypton plus the 14 5.5-neutron unit Xenon (8+14=22). Unlike the other noble gases, radon is unstable and yields radioactive isotopes if elements including plonium, lead and bismuth, it may be possible to form a stable Radon atom using the modeling to yield the stable/hybrid 22 5.5-6 unit neutron core.

Folding about a core traps information, overlap and information sharing (in chemistry this is paired electrons along fractal spiral overlays) between pair halves explains how bonding works, energy is pretime change.

Folding and unfolding effects may be maximized in terms of folding levels of compression to maximize magnetic, non-magnetic, energy storage, energy generation or other effects related to changing matrices.

As with iron exposed to a shaping field of ct4t11 states, the layout of individual atoms may be mechanically shaped and aligned to hold the desired effect. Any goals of alignment and the means to maximize one or more shapes in matrices lie in the designs and ability to affect them with KFE. Different protons and electrons can be targeted with KFE, although those most exposed to reaction at the ends of atoms have the more exposed electrons and energy applied to center of an atom would tend to flow out spontaneously due to balance to the ends or break the atom up in extreme cases, radioactive decay being an example, where the neutron core is imbalanced enough to be separated.

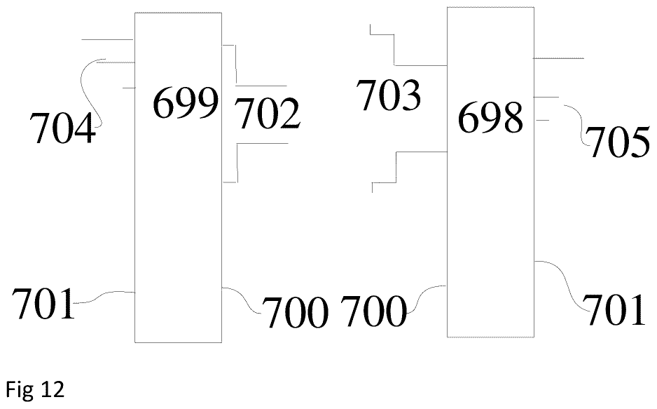

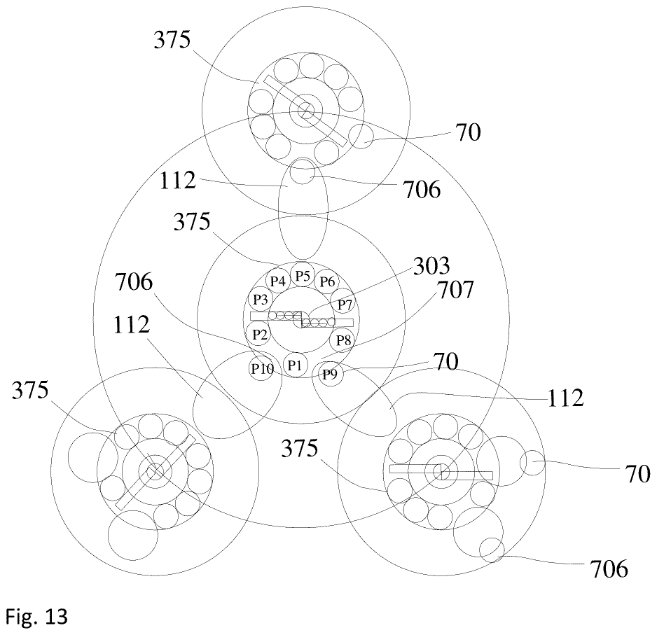

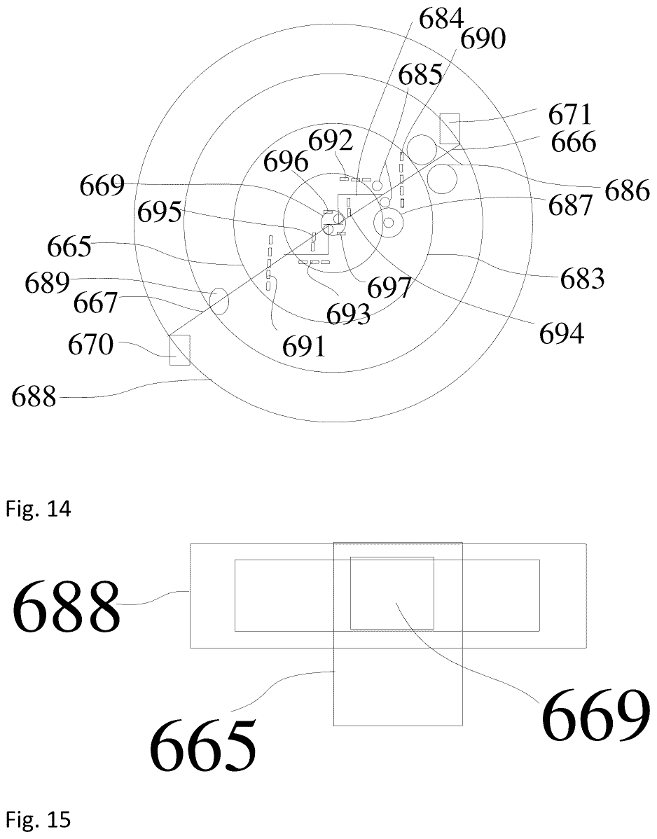

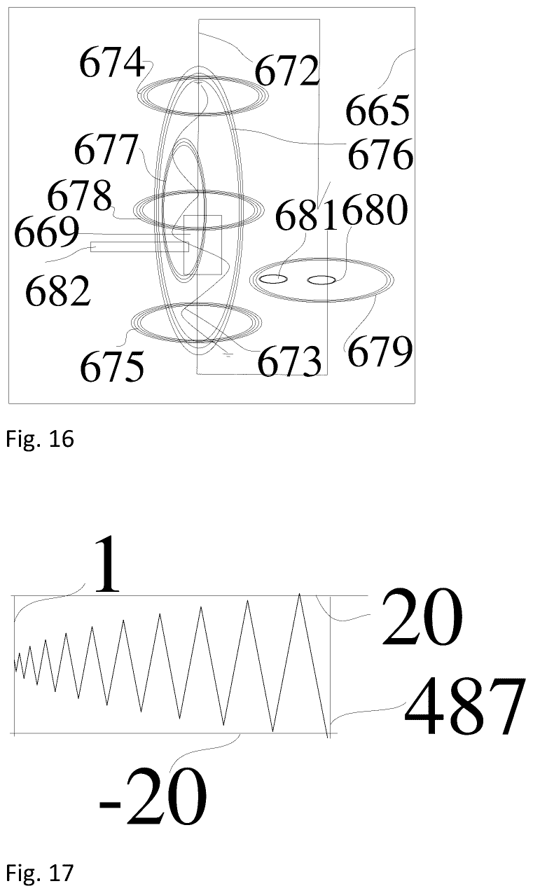

FIG. 12 shows how these can be addressed by, for example, changing the energy, spacing, and/or spiral or areas according to fractal features. Positive Electrode 698 (cathode) and negative electrode 699 (anode) have a front side 700 facing the opposite electrode and rear side 701 so that positive electrode extensions 703 can be designed to pull in neutron, MI based materials on the front and on the rear use the same type characteristics to encouraged oxygen to oxygen bonding while the negative electrode extensions 702 target the fpix based hydrogens with 1, 3, 5 modeling and 13 likewise encourage H2 bonding on the back so that one side can target disruption of bonding and the other side target encouraging bonding or one side can do both although they are separated here for clarity.

Exponential spacing, fpix spacing or MI spacing of multiple anodes and electrodes can be used to find the optimum relationship for different types of electrolysis.

Targeting hydrogen to hydrogen bonds involves fpix modeling. Targeting oxygen to hydrogen bonds involves fpix and MI modeling and oxygen to oxygen bonds involves fpix and MI but with more MI than the hydrogen oxygen bond which has one half operating 21 with fpix(hydrogen) and the other operating with MI and balancing fpix(oxygen). 22 The shaping of the electrodes, e.g., round to match 2\hat{ }n, hexagonal to match fpix or MI transitions at various stages can be used.

There are multiple places to disconnect so designing for each, one is the hydrogen bonds which are remotely shared between oxygen, a balance with a comparable “AuT-positron” equivalent coming off the Oxygen atom; and the second is that sharing more directly with oxygen. This process can be effective at the surface of a catalyst. 28 Starting with an initial separation through introduction of one or more type of information followed by a change to a second frequency or concentration of information.

Another item is inserting an information state within the matrix and then exciting those inserted states.

Fractal modeling: Fractal modeling is a mathematical technique that can be used to describe the structure of complex systems. In the context of hydrogen separation, fractal modeling can be used to identify the specific bonds between oxygen and hydrogen that are most susceptible to disruption. This information can then be used to design a separation process that targets these bonds and produces high-purity hydrogen.

CT state targeting: CT states are a type of quantum state that is associated with the overlap of electron orbitals. In the context of hydrogen separation, CT state targeting can be used to strengthen the bonds between oxygen and hydrogen that are desired, while weakening the bonds that are not desired. This can be done by applying an electric field or a magnetic field to the water molecules.

O—O sharing and H-H sharing: O—O sharing and H-H sharing are two types of chemical bonds that can form between oxygen and hydrogen atoms. O—O sharing is a type of covalent bond, while H-H sharing is a type of hydrogen bond. Fractal modeling can be used to identify the specific conditions that are necessary for O—O sharing and H-H sharing to occur. This information can then be used to design a separation process that produces high-purity hydrogen.

The following are advantages:

Fractal modeling is a powerful tool that can be used to describe the structure of complex systems.

CT state targeting can be used to strengthen or weaken specific bonds between atoms.

O—O sharing and H-H sharing are two types of chemical bonds that can be used to produce high-purity hydrogen.

Overall, the hydrogen separation techniques can incorporate these KFE for separation whether in membranes, steam reforming steps or within atomic or molecular structures designed as catalysts to lower the energy necessary for high-purity hydrogen.

The use of fractal features in the design of the electrodes can help to improve the efficiency of the separation process. Fractals are self-similar patterns that repeat at different scales, and this property can be used to create electrodes with a high surface area to volume ratio. This increased surface area will allow for more efficient contact between the electrodes and the water molecules, which will lead to a more complete separation of the hydrogen and oxygen.

The use of diverse types of information can also be used to improve the efficiency of the separation process. For example, one type of information that could be used is the frequency of the light that is used to illuminate the water. The frequency of the light can be chosen to match the resonant frequency of the hydrogen bonds, which will make it easier to break these bonds and separate the hydrogen and oxygen. Another way to improve the efficiency of the separation process is to insert an information state within the matrix. This can be done by using a laser to create a pattern of light within the water. The pattern of light can be used to create a specific energy state within the water, which will make it easier to separate the hydrogen and oxygen. Hydrogen separation techniques involve the separation of hydrogen from other elements or compounds, such as water. In the figure mentioned, hydrogen is generated from water using a fractal model that targets the bonds between oxygen and hydrogen.

Fractal modeling focuses on CT state sharing and disruption to separate hydrogen bonds and proton sharing.

Fractal patterns exist in the bonds and electron orbitals, which can be targeted to strengthen one type of hydrogen bond over the other. The fractal features change along fractal lines according to the fractal equations of FIP, allowing for efficient changes in the hydrogen bonds.

The location of where the charges are directed can be based on the MI, fpix, and 2\hat{ }n separation to mimic, replace, or disrupt different bonds based on their separation, information shared, base numbering, and other KFE.

Overall, hydrogen separation techniques involve targeting the bonds between hydrogen and other elements or compounds using fractal modeling to efficiently separate hydrogen bonds and proton sharing.

The separation of hydrogen from water using fractal modeling can be further optimized by manipulating the layout of the electrodes and the energy, spacing, and spiral or area according to fractal features. Electrodes with exponential or fractal spacing at different levels can be used to energize and separate hydrogen at different levels. The shaping of electrodes can also be used to match the transitions at different stages.

Multiple places to disconnect can be designed for, such as hydrogen bonds remotely shared between oxygen and sharing more directly with oxygen. This process can be effective at the surface of a catalyst.

To initiate separation, one or more types of information can be introduced followed by a change to a second frequency or concentration of information. Additionally, inserting an information state within the matrix and then exciting those inserted states can also be used.

Overall, manipulating the layout of electrodes and fractal features can optimize the separation of hydrogen from water and increase efficiency.

### Quantum Computing

To accomplish the goals, we compare how quantum processors approach probability modeling with fractal quantum change. In using pretime change, it need not be exclusive, but instead can be used to correct probability features of pretime computing to make the results more useful.

Time is stop-frame animation based on changes in the location of fractal pretime information states. The model of information physics used to quantify QC processors is appropriate to information (programming) based inquiries. Electrons are made up of photons in this scenario and the rotation of photon elements, even smaller pretime states than photons, around those photons when viewed from the standpoint of time appear as waves. Quantum computers mimic pretime computing using these pretime locations, but instead of using specific pretime locations to do computing, quantum computers use approximations based on probability. The resulting calculations have errors which limit the utility. If pretime positions are used, accurate results would be reached. Probabilistic results can be moderated with approximations of pretime positions.

Not all calculated positions need be precise. Instead, the pretime change analysis can be used to correct errors in probability modeling.

Qubit behavior can be predicted by analyzing pretime information state changes and information physics.

To achieve our objectives, we propose the following research activities:

By identifying the key factors that limit the performance of quantum computers and break them down into fractal components (or fractal equivalents which need to be substituted for those key factors) and using the mathematics of pretime dimensional change leading to post-time qubit and time related transitions, to reduce errors and get as close as possible to true “pretime” computing.

Noise is eliminated by coincidental or focused use of the concept of pretime computing to redefine our understanding of time and its role in quantum computing. Pretime information states, which are made up of photons and even smaller pretime states, can be used to predict the behavior of electrons. Quantum computers perform pretime computing, but approximations based on probabilities are used instead of the more precise pretime locations.

Through a reverse process, we can use quantum fractal algorithms and then look at the quantum computing results to find corrections, using known fractal results to correct the quantum errors, or in this case to see how closely probability-based devices come to getting accurate results.

This is an example of using a fractal framework for assessing the utility of quantum processors.

QC relies on true randomness. Qubits (electrons for example) are composites of fpix bits, fractal components changing according to set rules which occur in a pretime environment.

Errors can be corrected by applying pretime analysis of the changing bits allowing the speed of quantum computing with a “correcting” factor tied to the pretime changes in position which are otherwise relegated to random approximation. A crude attempt at using Fibonacci spirals had positive results in Quantum Computing. These crude experiments highlight the difference between understanding cause and effect and just recognizing the effect; but it shows the science can be used to manipulate qubits.

### Communication and Time

FIP involves using the relationships of CT state exchange along KFE features of information capture and release to more efficiently interpret results and increase post time speeds by concentrating on pretime CT states which allow for more instantaneous communication and computing.

Quantum measurements include replacing Planck length with quantum CT state changes at least one CT state compression level. The method includes tracking 1) absolute time and 2) utilizing the rate of absolute change for time keeping, reconciling electromagnetic time (EMT) with absolute time, using quantum change to determine pretime wave characteristics, and converting existing clock models to quantum change some of which affect time and some of which are pretime.

Perceived Time is stop-frame animation effects, changes are in fractal building blocks of electrons, ct412 states which, in turn, are made up of 10 ct4t11 states, typically viewed as photons. The electron ct4t12 states and lower components (ct4t11 states) are in multiple locations and states for any measurable time and appear relativistic but only from a perspective of time. By dimensional analysis they remain Newtonian. Focusing on CT state pretime locations, quantum computing is done without or with at least less “probability” which suggests a “false randomness” which is replaced with specific “pretime locational or net FIP state change where FIP state changes are the positive or negative states in which ct1 states find themselves which are reflected in fractally significant positive or negative states in higher compression states. Pretime locations are locations where a particle exists from a pretime environment as seen from the perspective of time. Since these can be significant for any period (practical measurements of 10\hat{ }44 of these changes per second) pretime change computing is exponentially higher than with other computers modes.

### Time Dilation

1) Time Dilation: The ratio of pre-ct4t13 states passing within a ct4-ct5 transitional state to pre-ct4t13 states (PTS) changing outside of the ct4-ct5 transitional state is a time dilation ratio. This ratio of PTS to PTS moving in and out is the source of velocity time dilation. The movement of PTS within alter the arrangement of the wave states captured between the proton and the electron altering the history of points within the transitional state and the comparison of one collection of points to subsequent 27 arrangements of the same points creates history.

### 2) Chapter 71 Relativity

To understand this, we start with fuse length and folding. At the edges of the universe the fuse length for newly created particles is 2 counts, then 4, the 6, and so on. At the center of the galaxy, the fuse lengths are older, on a scale of 10\hat{ }150 counts. Things at the center of the universe look amazingly stagnant, things at the edges change so fast they are pretime.

Two things happen with acceleration. 1) time slows down and 2) length increases. Time is change at the ct4t10-13 range, everything before that is pretime change that gives rise to time. More pretime change, less time since the pretime change is just more movement relative to post-time change. Acceleration is the change rate between time and pretime changes. Everything changing faster is invisible, everything changing slower is visible from the standpoint of time. All points exist frozen between quantum counts, having neither time nor speed. But as the quantum count increases certain points change and up to the ct4t12 state, these changes are acceleration, whether they are compressive or decompressive.

If you take two Matrix A (Still 1) and B (Still) which are identical and place them equal distance from the center of the universe we can see how this works. As change occurs within the matrix at the pretime level relative to the center (in this case accelerating away) there is more pretime change so the time within the point begins to change slower.

Likewise, the more solid part of the matrix begins to expand from the standpoint of time because each point that is changing in a pretime environment appears to be more places at once, hence appearing to assume more space, the item lengthens.

Acceleration has two components. 1) unfolding or folding change relative to a universal center and 2) pretime change compared to post-time change. Whether you accelerate towards or away from the center, the amount of ct1 change increases so that more of the change is pretime slowing down time while increasing the change within the system.

Gravity can be seen as pulling CT states within the matrix represented by the larger circle. Put another way, gravity is a net increase in folding. The more change occurs in compressed matrix (ct4t12 and above) the more the change is visible from the standpoint of time. With sufficient folding, since more of the ct1 states are part of CT states that are higher than ct4t12, the slower time goes for a given set of points because less of them are changing pretime creating more frames.

The changes expanding and contracting the size of the matrix appear as work and this is why we see energy as energy.

Dimension is also affected. As an increasing number of the ct2s straighten the entire matrix begins to flatten out. At an inflection point ct4 breaks down first from neutrons to partially filled states (e.g., electrons and protons) and then into energy.

1. A method for improving processes utilizing Key Fractal Elements (KFE), from the group comprising (1) Applying KFE to affect change in CT states and interpret or categorize matrices of CT states (2) Utilizing KFE elements for categorization, prediction, manipulation, and designing applications, (3) Employing KFE to model ct state transitions for desired efficiencies and results, (4) Leveraging KFE elements to govern matrix composition and interaction, encompassing spatial, energy, atomic, chemical, electrical, biological, and large structures, (5) Utilizing KFE to model compression or decompression of CT states and matrices of CT states, facilitating desired dimensional changesm (6) Designing matrices based on KFE concepts, considering time as CT state dimensional change, (7) Incorporating KFE to facilitate the design and manipulation of molecules, including the expansive or contractive features and orientation of atomic or molecular matrices throughout reactions as CT state matrix change occurs, (8) Controlling the absorption and spew of CT state exchanges within matrices by strategically shaping reaction chambers according to KFE principles, (9) Applying KFE to react chemicals for enhanced matrix changes, maximizing the release of pretime informational change, and optimizing energy utilization, (10) Employing KFE to target FIP features of stepped transitions, categorization, chemistry, biology, and other relevant features, (11) Leveraging KFE to achieve balance within matrices, utilizing plasma fulcrums, absorption, and spew mechanisms, and leveraging fractal alignment patterns for efficient CT state interactions, (12) Utilizing KFE to design, control, and optimize the proximity of states, the nature of intervening matrices, and the mixing of CT states for desired matrix results.

2. The invention of claim 1 wherein the KFE are based on at least one of the following: fpix, resonance based on fpix, 2\hat{ }n compression, 2f(n)\hat{ }(2\hat{ }n) compression where f(n) is equal to fpix or MI based on the level of compression separated at neutron compression, and overlapping MI spirals associated with 2\hat{ }n compression.

3. The method of claim 2 directed towards a method for using fractals with data science to modify AI software to maximize the accessibility, organization, and value of empirical data and to allow this data to be used along with fractal modeling to predict chemical and biological functions, the method comprising the steps of:
a. Providing a fractal model applicable across energy, atomics, chemical and biological systems, categorizing data from the sub-atomic through the molecular systems up to and including complex biological systems and astronomic features allowing data across those areas to be categorized using the same modeling;
b. Developing a classification system to cross-characterize existing, empirical non-fractal data within sub-atomic, atomic, chemical, and biological data with fractal modeling required by the KFE Fractal modeling;
c. Integrating the fractal model into AI software to allow the AI software to use the fractal model to access, organize, and analyze empirical data; and
d. Using the AI software to predict at least one of electromagnetic, chemical and biological functions.

4. The invention of claim 2 wherein the invention further comprises a system for automated fractal analysis of empirical chemistry data, the system comprising:
a database storing scientific data related to atomic interactions; a software module configured to: access the empirical chemistry data; extract features from the data and categorize them based on fractal constraints; generate fractal models predicting transitions between energy and matter based on categorized data; perform simulations using the fractal models; compare simulation results to empirical data;
identify discrepancies between empirical data and simulation predictions; and refine the fractal models to reconcile differences with empirical data.

5. The invention of claim 4 further comprising classification system to cross-characterize existing, empirical non-fractal data within sub-atomic, atomic, chemical, and biological data with fractal modeling based on the KFE.

6. The method of claim 2 applied to modeling atomic interactions for energy transfer, the method comprising:
a. selecting empirical data related to atomic interactions for one or more energy processes;
b. designing all matrices from the empirical data based on KFE;
c. developing fractal atomic structure definitions and mathematical models describing transitions between energy and matter based on KFE;
d) using KFE to refine the fractal models to make them more efficient.

7. The method of claim 6 further comprising improving fusion processes using Key Fractal Elements (KFE), comprising utilizing KFE to affect change in CT states, interpret matrices of CT states, and design radiation matrices for fusion energy production, wherein the application of KFE enables enhanced fusion reactions, efficient energy release, and improved control over the fusion process.

8. The method of claim 6 further comprising improving fission processes using Key Fractal Elements (KFE), comprising utilizing KFE to affect change in CT states, interpret matrices of CT states, and optimize fission reactions, wherein the application of KFE enables efficient and controlled fission reactions, enhanced energy generation, and improved safety measures.

9. The process of claim 8 further comprising: a fission chip comprising multiple layers wherein the layers including at least one fission means for generating energy from a fission reaction; a conversion means for converting radiation into at least one desired pretime change concentration using KFE; at least one collection means to collected the at least one pretime change concentration using KFE; and at least one transfer means for exporting the collected pretime change concentration outside of the chip using KFE.

10. The invention of claim 9 wherein the KFE include modeling the chemical elements and molecules used to maximize each step utilizing transitions defined by KFE to design the fission means to maximize the efficiency of the entire chip; separate ct states as desired, to funnel ct states to the collecting means, to place the collecting means relative to the fission means; and to place and utilize the transfer means most efficiently.

11. The method of claim 2 further comprising improving chemistry processes using Key Fractal Elements (KFE), comprising utilizing KFE to affect change in CT states, interpret matrices of CT states, and optimize chemical reactions, wherein the application of KFE enables enhanced reaction kinetics, increased reaction selectivity, and improved product yields.

12. A process for practicing fractal science comprising:
providing a fractal model applicable across energy, atomic, chemical, and biological systems; developing a classification system to cross-characterize existing empirical non-fractal data within sub-atomic, atomic, chemical, and biological domains with the fractal modeling; integrating the fractal model into artificial intelligence (AI) software;
using the AI software to access, organize, and analyze empirical data;
predicting chemical and biological functions based on the analyzed data;
selecting empirical data related to atomic interactions for various applications;
analyzing the empirical data and identifying patterns through fractal analysis techniques; developing fractal atomic structure definitions and mathematical models describing transitions between energy and matter based on the identified patterns; performing simulations using the developed models to predict the behavior of fractal particles during dimensional transformations; comparing simulation predictions to empirical results; and refining the fractal models based on comparison to empirical data.

13. The process as claimed in claim 12, wherein the fractal model is based on fpix, resonance based on fpix, 2\hat{ }n compression, 2f(n)\hat{ }(2\hat{ }n) compression where f(n) is equal to fpix or MI based on the level of compression separated at neutron compression, and overlapping MI spirals associated with 2\hat{ }n compression.

14. The process as claimed in claim 12, further comprising a classification system cross-characterizes existing empirical data with the fractal modeling.

15. The process as claimed in claim 13, wherein the system for automated fractal analysis of empirical chemistry data comprises:
a database that stores empirical chemistry data related to atomic interactions;
a software module configured to access the empirical chemistry data, extract features from the data, and categorize them based on fractal constraints;
a processor configured to execute the software module.

16. The process as claimed in claim 15, wherein the software module generates fractal models predicting transitions between energy and matter based on categorized data, performs simulations using the fractal models, compares simulation results to empirical data, identifies discrepancies between empirical data and simulation predictions, and refines the fractal models to reconcile differences with empirical data.

17. The process as claimed in claim 13, wherein the fractal model is used to improve energy, fission and fusion processes by utilizing Key Fractal Elements (KFE) to affect change in CT states, interpret matrices of CT states, and design chemical, plasma, and radiation matrices to optimize chemical and energy transitions.

18. A method for analyzing and manipulating information states defined as parts dimensional features comprised of combined solutions of fpix to design electromechanical, physics and chemical interactions in structural components, and wherein combing solutions is compression and breaking down combined solutions is decompression of dimension comprising:
a. Identifying the KFE fractal information content of dimensional manifestations using a fractal-based model, on the observed bit fpix up to neutrons;
b. Identifying the KFE fractal information content of atomic dimensional manifestations using a fractal-based model, on the geometry of MI beginning at neutron bonding;
c. applying the KFE fractal information content to analyze and manipulate matrices of fractal states defined using fpix and dimensional transitions.

19. The method of claim 18 further comprising a method for enhancing energy conversion efficiency, comprising:
a. configuring elements control reactions and electromagnetic dimensional transitions between a pi-defined geometry and a MI-defined geometry at neutron bonding; wherein the fractally mandated dimensional transitions based on key fractal elements from the set comprising 2f(n), n=n+1, particularly to 3 places, fpix, Fibonacci (MI) or MI/fpix transitions, and wherein f(n) is from the group comprising fpix and MI, and 2\hat{ }n steps of intermediary compression steps and where force represents the exchange of information states.

20. The method of claim 18 wherein the dimensional manifestations are from the group comprising the process of designing fractal changes to enhance a process from the group consisted of AI algorithms, energy manipulation, material manipulation, energy storage, energy generation, machine learning, chemistry, predictive science, and quantum computing.
